(19) United States
(12) Patent Application Publication
(10) Pub. No.: US 2026/0297024 A1
(43) Pub. Date: Oct. 1, 2026

(54) PROCESSES AND SYSTEMS FOR FORMING FUNCTIONALIZED ORGANIC SULFIDES

(71) Applicant: Chevron Phillips Chemical Company LP, The Woodlands, TX (US)

(72) Inventors: Christophe LAROCHE, Bartlesville, OK (US); Michael DABORN, Headley (GB); Jasper SMETS, Antwerpen (BE); Nicolas BLOUIN, Grez-Doiceau (BE); Kenneth M. LASSEN, Bartlesville, OK (US); Freddy KLEIS, Knokke-Heist (BE)

(21) Appl. No.: 19/577,231

(22) Filed: Mar. 24, 2026

Related U.S. Application Data
(60) Provisional application No. 63/779,993, filed on Mar. 28, 2025.

(51) Int. Cl. C07C 319/18; B01J 19/00

(52) U.S. Cl. CPC C07C 319/18; B01J 19/0006

(57) ABSTRACT

Disclosed herein are new processes for forming functionalized organic sulfides. Also disclosed herein are systems for forming functionalized organic sulfides and uses of the functionalized organic sulfides. In an aspect, a process for forming a functionalized organic sulfide is provided. The process includes introducing an olefin to a reactor and introducing a mercaptan to the olefin to form a mixture. The process further includes reacting the mixture, under conversion conditions, to form a conversion product effluent comprising a functionalized organic sulfide and unreacted olefin. The process further includes separating the unreacted olefin from the conversion product effluent.

### CROSS-REFERENCE TO RELATED APPLICATIONS

This application claims benefit of and priority to U.S. Provisional Patent Application Ser. No. 63/779,993, filed on Mar. 28, 2025, which is incorporated herein by reference in its entirety.

### FIELD

Aspects of the present disclosure generally relate to new processes for forming functionalized organic sulfides and to new systems for forming functionalized organic sulfides. Aspects of the present disclosure also generally relate to uses of the functionalized organic sulfides.

### BACKGROUND

Organic sulfides, also referred to as thioethers (R′—S—R″), are used in a variety of applications including mining chemical collectors. Conventionally, organic sulfides are prepared by alkylation of thiols using alkyl halides. However, the use of alkyl halides results in formation of corrosive and toxic hydrobromic acid (HBr) or hydrochloric acid (HCl), potentially generating molecular bromine (Br₂) or chlorine (Cl₂). The safety and post-synthesis work-up protocols render this method too time intensive and hazardous. Another conventional method involves reaction of disulfides with organolithium reagents or Grignard reagents. Organolithium reagents and Grignard reagents are also hazardous and their use requires extensive precautions. Besides these drawbacks, this organometallic method requires starting from a disulfide molecule. On large scales, these conventional methods are too costly, time consuming, and hazardous. Another conventional method to form organic sulfides is a thiol-ene reaction between a thiol and an alkene. However, such reactions require a photoinitiator (with light), a high concentration of radical initiator, or high heat to react the alkene with the thiol. In addition to the disadvantages of the aforementioned synthetic methods, conventional synthetic methods also require solvents or unstable radical initiators such as peroxides or azobisisobutyronitrile (AIBN). Oftentimes, radical initiators are not compatible with functional groups such as alcohols, esters, and carboxylic acids, among other functional groups.

There is a need for new processes for forming functionalized organic sulfides. There is also a need for new systems for forming functionalized organic sulfides.

### SUMMARY

Aspects of the present disclosure generally relate to new processes for forming functionalized organic sulfides, to new systems for forming functionalized organic sulfides, and to uses of the functionalized organic sulfides. The inventors found novel processing schemes for forming functionalized organic sulfides from olefin and mercaptan starting materials. Advantageously, and unlike conventional methods, aspects described herein enable the formation of functionalized organic sulfides without the addition of catalyst or energy source (light, heat, or both). When heat is used as an energy source, formation of functionalized organic sulfides can proceed at temperatures of about 100° C. or less, or even at 20° C. or 25° C. Further, formation of functionalized organic sulfides according to aspects of the present disclosure is tolerant to, and compatible with, a broad scope of functional groups. In addition, unlike state-of-the-art methods, aspects described herein enable formation of functionalized organic sulfides without the use of solvents.

In an aspect, a process for forming a functionalized organic sulfide is provided. The process includes introducing, to a reactor, an olefin represented by formula (I):

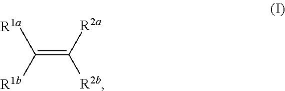

wherein each of R^{1a}, R^{1b}, R^{2a}, and R^{2b} of formula (I) is, independently, hydrogen, a C1-C20 unsubstituted hydrocarbyl, a C1-C20 substituted hydrocarbyl, or two or more of R^{1a}, R^{1b}, R^{2a}, or R^{2b} join together to form a C4-C30 monocyclic or polycyclic ring structure. The process further includes introducing a mercaptan to the olefin represented by formula (I) to form a mixture, the mercaptan represented by formula (II):

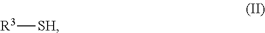

wherein R³ of formula (II) is a hydrogen, a C1-C24 substituted hydrocarbyl, or a C1-C24 unsubstituted hydrocarbyl. The process further includes reacting the mixture, under conversion conditions, to form a conversion product effluent comprising a functionalized organic sulfide and unreacted olefin represented by formula (I). The process further includes separating the unreacted olefin from the conversion product effluent.

In another aspect, a solvent-free process for forming a functionalized organic sulfide is provided. The process includes heating or maintaining, in a reactor, an olefin at a temperature in a range from about 20° C. to about 180° C., the olefin represented by formula (I):

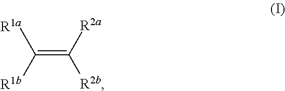

wherein: each of R^{1a}, R^{1b}, R^{2a}, and R^{2b} of formula (I) is, independently, hydrogen, a C1-C20 unsubstituted hydrocarbyl, a C1-C20 substituted hydrocarbyl, or two or more of R^{1a}, R^{1b}, R^{2a}, or R^{2b} join together to form a C4-C30 monocyclic or polycyclic ring structure. The process further includes introducing a mercaptan to the olefin represented by formula (I) to form a mixture, the mercaptan represented by formula (II):

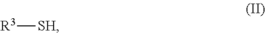

wherein R³ of formula (II) is a C1-C24 substituted hydrocarbyl; and wherein a molar ratio of the olefin represented by formula (I) to the mercaptan represented by formula (II) in the mixture is within a range from about 1:1 to about 10:1. The process further includes reacting the mixture, under conversion conditions, to form a conversion product effluent comprising a functionalized organic sulfide and unreacted olefin represented by formula (I), the conversion conditions comprising: exposing the mixture to a partial pressure of a first gas comprising air, O₂, or a combination thereof, and exposing the mixture to a partial pressure of a second gas that is different from the first gas, the second gas comprising a non-reactive gas. The process further includes separating the unreacted olefin from the conversion product effluent. The process further includes combining the unreacted olefin with the mercaptan represented by formula (II) in the reactor.

In another aspect, a system for forming a functionalized organic sulfide is provided. The system includes a reactor configured to receive a first feed comprising an olefin, to heat the first feed, to receive a second feed comprising a mercaptan, to react a mixture comprising the first feed and the second feed under conversion conditions, and to discharge a conversion product effluent comprising a functionalized organic sulfide and unreacted olefin. The system further includes a separation unit configured to receive the conversion product effluent and to separate the conversion product effluent into a plurality of output streams, a first output stream of the plurality of output streams comprising the unreacted olefin.

In another aspect, a composition is provided that includes a functionalized organic sulfide described herein.

In another aspect, a mining chemical collector is provided. The mining chemical collector includes a functionalized organic sulfide described herein.

### BRIEF DESCRIPTION OF THE DRAWINGS

So that the manner in which the above recited features of the present disclosure can be understood in detail, a more particular description of the disclosure, briefly summarized above, can be had by reference to aspects, some of which are illustrated in the appended drawings. It is to be noted, however, that the appended drawings illustrate only exemplary aspects and are therefore not to be considered limiting of its scope for the disclosure can admit to other equally effective aspects.

FIG. 1 is a flow diagram showing selected operations of a process for forming a functionalized organic sulfide according to at least one aspect of the present disclosure.

FIG. 2 is a generalized schematic flow diagram showing various implementations of processes described herein corresponding to operational areas or units in a system for forming a functionalized organic sulfide according to at least one aspect of the present disclosure.

FIG. 3 is a reaction diagram showing a proposed mechanism for forming a functionalized organic sulfide according to at least one aspect of the present disclosure.

To facilitate understanding, identical reference numerals have been used, where possible, to designate identical elements that are common to the figures. It is contemplated that elements and features of one aspect can be beneficially incorporated in other aspects without further recitation.

### DETAILED DESCRIPTION

Aspects of the present disclosure generally relate to new processes for forming functionalized organic sulfides and to new systems for forming functionalized organic sulfides. Aspects of the present disclosure also generally relate to uses of the functionalized organic sulfides. A functionalized organic sulfide is an organic sulfide (containing carbon, hydrogen, and sulfur atoms only) that, besides the sulfur atom, has an additional functional group containing at least one heteroatom or heteroatom-containing group, such as one or more elements from Group 13-17 of the periodic table of the elements. For example, a functionalized organic sulfide includes: (a) a sulfur atom in the form of, for example, a sulfide (thioether); and (b) an additional functional group such as an alcohol functional group, an ester functional group, a carboxylic acid functional group, a nitrile functional group, an amine functional group, or an amide functional group, among other functional groups. The term “functionalized organic sulfide” can be used interchangeably with the term “functionalized thioether”.

Scheme A shows a general, and non-limiting, reaction diagram for forming functionalized organic sulfides according to aspects described herein. As shown in Scheme A, an olefin represented by formula (I) and a mercaptan represented by formula (II) react to form a functionalized organic sulfide represented by formula (X). R^{1a}, R^{1b}, R^{2a}, R^{2b}, and R³ are further described below.

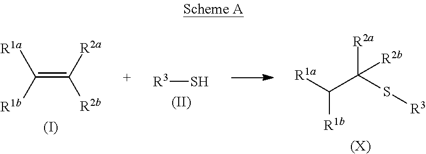

The mercaptan represented by formula (II) can add to the less substituted carbon of the olefin represented by formula (I) or to the more substituted carbon of the olefin represented by formula (I), producing a mixture of functionalized organic sulfide products. Addition to the less substituted carbon follows anti-Markovnikov addition, while addition to the more substituted carbon follows Markovnikov addition. In some implementations, which can be combined with other implementations, aspects described herein can form the anti-Markovnikov addition product as the major product of the reaction. Small amounts, non-detectable amounts, or traces of the Markovnikov addition product can optionally be formed with aspects described herein.

Advantageously, aspects described herein avoid the use of alkyl halides and therefore do not generate hazardous chemicals such as HBr, HCl, molecular bromine, or chlorine gas as observed with conventional alkylation of thiols. In addition, aspects of the present disclosure avoid the use of organomagnesium reagents (Grignard reagents) and organolithium reagents and are not dependent on a disulfide starting material.

Other conventional methods are silent on industrial scale processing, only being performed at small, laboratory scales. In addition, and unlike state-of-the-art technologies, aspects described herein can include a separation step on the conversion product containing the functionalized organic sulfide (for example, represented by formula (X)). The separation step enables removal of unreacted olefin which can then be recycled back to the reaction for forming more functionalized organic sulfide. Accordingly, aspects described herein enable large scale efficiencies not present in state-of-the-art technologies. In some aspects, which can be combined with other aspects, the reaction shown in Scheme A can be performed in the presence of a partial pressure of air and/or oxygen (O₂) and a partial pressure of a different gas such as a non-reactive gas. In some aspects, the conversion conditions can include exposing the reaction mixture to O₂ or a gas comprising O₂ (e.g., air). While not wishing to be bound by any theory, it is believed that O₂ can catalyze the reaction between the olefin represented by formula (I) and the mercaptan represented by formula (II). While not wishing to be bound by any theory, it is believed that O₂ can facilitate initiation or promotion of the thiol-ene addition reaction between the mercaptan represented by formula (II) and the olefin represented by formula (I). For example, O₂ can assist in the formation of thiyl radicals from the mercaptan, which can add across the olefin to form the corresponding thioether product. Accordingly, exposure to O₂ can promote or accelerate formation of the functionalized organic sulfide. In some aspects, which can be combined with other aspects, an O₂-containing gas can be present in an amount sufficient to promote initiation of the reaction while minimizing oxidative side reactions such as formation of disulfides from the mercaptan. In certain aspects, the mixture can additionally or alternatively be exposed to an inert or non-reactive gas (e.g., nitrogen), for example, to control reaction conditions, including the partial pressure of O₂ present in the reactor.

In further contrast to conventional methods, aspects described herein enable formation of functionalized organic sulfides without a solvent. For example, the inventors found that a solvent-free reaction mixture containing an olefin represented by formula (I) and a mercaptan represented by formula (II) can be reacted to form the functionalized organic sulfide. Other operations of processes described herein can also be solvent-free. The use of solvents, however, is contemplated. Solvent-free operations and processes described herein make aspects of the present disclosure more economical and less time intensive.

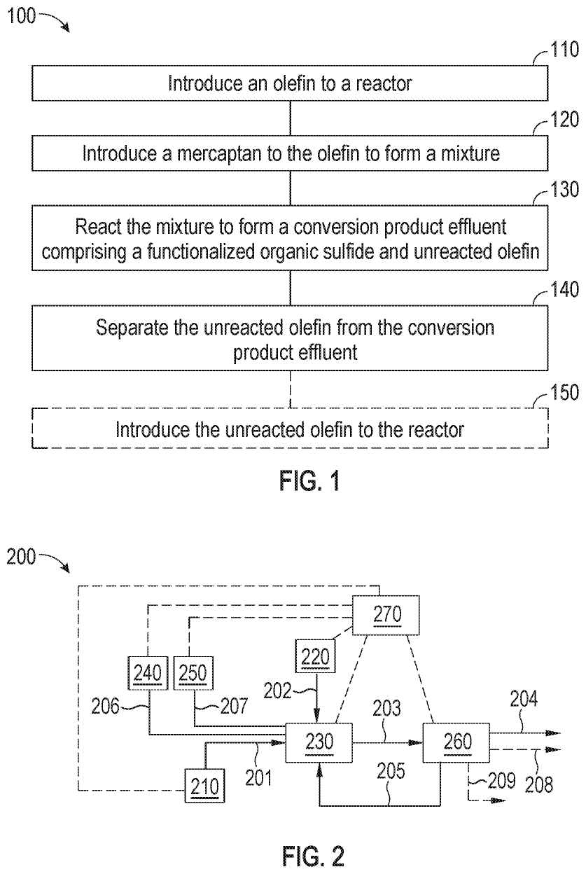

Aspects of the present disclosure generally relate to new processes for forming functionalized organic sulfides. FIG. 1 is a flow diagram showing selected operations of a process 100 for forming functionalized organic sulfides according to at least one aspect. Aspects and implementations of process 100 can be combined with other aspects and implementations described herein, such as aspects and implementations described with respect to FIG. 2. In general, processes of the present disclosure enable conversion of an olefin to various functionalized organic sulfides.

The process 100 can begin with introducing an olefin to a reactor at operation 110. The reactor can correspond to reactor 230 of FIG. 2 further described herein. The term “olefin” is used herein in accordance with the definition specified by IUPAC: acyclic and cyclic hydrocarbons having one or more carbon-carbon double bonds apart from the formal ones in aromatic compounds. The class “olefins” subsumes alkenes and cycloalkenes and the corresponding polyenes. Ethylene, propylene, 1-butene, 2-butene, 1-hexene, and the like are non-limiting examples of olefins. The term “alpha-olefin” as used herein refers to an olefin that has a double bond between the first and second carbon atom of the longest contiguous chain of carbon atoms. The term “alpha-olefin” includes linear and branched alpha-olefins unless expressly stated otherwise. 1-butene, 1-hexene, 1-octene, and the like are non-limiting examples of alpha-olefins.

Any suitable olefin can be used at operation 110, such as an olefin represented by formula (I):

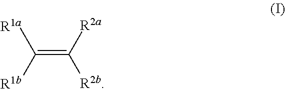

Each of R^{1a}, R^{1b}, R^{2a}, and R^{2b} of formula (I) can be, independently, hydrogen, an unsubstituted hydrocarbyl, a substituted hydrocarbyl, or two or more of R^{1a}, R^{1b}, R^{2a}, or R^{2b} join together to form a monocyclic or polycyclic structure. Each of R^{1a}, R^{1b}, R^{2a}, and R^{2b} of formula (I) can be the same or different. More than one olefin represented by formula (I) can be utilized at operation 110.

The term “hydrocarbyl group” is used herein in accordance with the definition specified by IUPAC: a univalent group formed by removing a hydrogen atom from a hydrocarbon. “Hydrocarbon” refers to a compound containing only carbon and hydrogen. Hydrocarbyl groups can be linear or branched, saturated or unsaturated, cyclic or acyclic, aromatic or non-aromatic. Non-limiting examples of hydrocarbyl groups can include alkyl, alkenyl, cycloalkyl, aryl, and aralkyl groups, amongst other groups, such as ethyl, phenyl, tolyl, propenyl, and the like.

An “unsubstituted hydrocarbyl” refers to a group that consists of hydrogen and carbon atoms only. “Unsubstituted hydrocarbyl” includes groups having C1-C100 groups and can be linear or branched, acyclic or cyclic, saturated or unsaturated. When cyclic, the unsubstituted hydrocarbyl can be aromatic or non-aromatic. Regarding saturation, the unsubstituted hydrocarbyl can be fully saturated, partially unsaturated, or fully unsaturated. Non-limiting examples of unsubstituted hydrocarbyl include a group having from 1 to 100 carbon atoms, such as from 1 to 30 carbon atoms, such as 1 to 20 carbon atoms, such as 3 to 10 carbon atoms, such as methyl, ethyl, n-propyl, isopropyl, n-butyl, iso-butyl, sec-butyl, and tert-butyl, pentyl, hexyl, heptyl, octyl, ethyl-2-hexyl, isooctyl, butyl-2-octyl, nonyl, n-decyl, isodecyl, or isomers thereof, a cycloaliphatic group having from 3 to 20 carbon atoms such as, for example, cyclopentyl or cyclohexyl; an aromatic group having from 6 to 20 carbon atoms such as, for example, phenyl or naphthyl; or any combination thereof.

A “substituted hydrocarbyl” refers to an unsubstituted hydrocarbyl in which at least one hydrogen atom of the unsubstituted hydrocarbyl has been substituted with at least one heteroatom or heteroatom-containing group, such as one or more elements from Group 13-17 of the periodic table of the elements, such as halogen (F, Cl, Br, or I), O, N, Se, Te, P, As, Sb, S, B, Si, Ge, Sn, Pb, and the like, such as C(O)R*, C(C)NR*2, C(O)OR*, NR*2, OR*, SeR*, TeR*, PR*2, AsR*2, SbR*2, SR*, SOx (where x=2 or 3), BR*2, SiR*3, GeR*3, SnR*3, PbR*3, and the like, where R* is, independently, hydrogen or unsubstituted hydrocarbyl, or where at least one heteroatom or heteroatom containing group has been inserted within the unsubstituted hydrocarbyl such as —O—, —S—, —Se—, —Te—, —N(R*)—, ═N—, —P(R*)—, ═P—, —As(R*)—, ═As—, —Sb(R*)—, ═Sb—, —B(R*)—, ═B—, —Si(R*)₂—, —Ge(R*)₂—, —Sn(R*)₂—, —Pb(R*)₂— and the like, where R* is independently a hydrocarbyl radical, and two or more R* can join together to form a substituted or unsubstituted, fully saturated, partially unsaturated, or fully unsaturated, aromatic or non-aromatic C4-C62 monocyclic or polycyclic ring structure.

Referring back to the olefin represented by formula (I), each of R^{1a}, R^{1b}, R^{2a}, and R^{2b} of formula (I) can have any suitable number of carbon atoms, for example, 1 to 30 carbon atoms. Each of R^{1a}, R^{1b}, R^{2a}, and R^{2b} of formula (I) can be, independently, hydrogen, a C1-C20 unsubstituted hydrocarbyl, a C1-C20 substituted hydrocarbyl, or two or more of R^{1a}, R^{1b}, R^{2a}, or R^{2b} join together to form a monocyclic or polycyclic ring structure.

Each of R^{1a}, R^{1b}, R^{2a}, and R^{2b} of formula (I) can be, independently, linear or branched, saturated or unsaturated, cyclic or acyclic, monocyclic or polycyclic, aromatic or non-aromatic. Regarding saturation, each of R^{1a}, R^{1b}, R^{2a}, and R^{2b} of formula (I) can be, independently, fully saturated, partially unsaturated, or fully unsaturated.

Each of R^{1a}, R^{1b}, R^{2a}, and R^{2b} of formula (I) can be, independently, hydrogen, a linear C1-C20 unsubstituted hydrocarbyl, a branched C1-C20 unsubstituted hydrocarbyl, a cyclic C1-C20 unsubstituted hydrocarbyl, an acyclic C1-C20 unsubstituted hydrocarbyl, or combinations thereof. Suitable C1-C20 unsubstituted hydrocarbyls include methyl, ethyl, propyl, iso-propyl, butyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl. Isomers, such as linear, branched, acyclic, and cyclic isomers of the aforementioned C1-C20 unsubstituted hydrocarbyls are contemplated.

Each of R^{1a}, R^{1b}, R^{2a}, and R^{2b} of formula (I) can be, independently, a C1-C18 unsubstituted hydrocarbyl, such as a C2-C16 unsubstituted hydrocarbyl, such as a C4-C16 unsubstituted hydrocarbyl, such as a C5-C12 unsubstituted hydrocarbyl, such as a C6-C10 unsubstituted hydrocarbyl.

Each of R^{1a}, R^{1b}, R^{2a}, and R^{2b} of formula (I) can be, independently, a C1-C18 substituted hydrocarbyl, such as a C2-C16 substituted hydrocarbyl, such as a C4-C16 substituted hydrocarbyl, such as a C5-C12 substituted hydrocarbyl, such as a C6-C10 substituted hydrocarbyl.

When two or more of R^{1a}, R^{1b}, R^{2a}, or R^{2b} join together to form a monocyclic or polycyclic ring structure, the monocyclic or polycyclic ring structure can be substituted or unsubstituted, fully saturated, partially unsaturated, or fully unsaturated, aromatic or non-aromatic. When two or more of R^{1a}, R^{1b}, R^{2a}, or R^{2b} join together to form a monocyclic or polycyclic ring structure, the monocyclic or polycyclic ring structure can be a C4-C40 monocyclic or polycyclic ring structure, such as a C4-C30 monocyclic or polycyclic ring structure, such as a C4-C20 monocyclic or polycyclic ring structure, such as a C5-C20 monocyclic or polycyclic ring structure, such as a C5-C14 monocyclic or polycyclic ring structure, such as a C6-C12 monocyclic or polycyclic ring structure. For example, R^{1a} and R^{2a} of formula (I) can join together to form a monocyclic or polycyclic ring structure, for example a C4-C30 monocyclic ring or a polycyclic ring. As an illustrative, but non-limiting, example, R^{1a} and R^{2a} can join together to form a ring, such as a cyclohexene. As another non-limiting example, norbornene can be utilized as an olefin represented by formula (I).

Each of R^{1a} and R^{1b} of formula (I) can be, independently, hydrogen, or a linear or branched, cyclic or acyclic C2-C16 unsubstituted hydrocarbyl; and each of R^{2a} and R^{2b} of formula (I) can be, independently, hydrogen, methyl, or ethyl.

Compounds that include more than one unsaturated carbon-carbon bond are contemplated, such as dienes, trienes, etc. Accordingly, each of R^{1a}, R^{1b}, R^{2a}, and R^{2b} of formula (I) can contain, independently, at least one unsaturated carbon-carbon bond, for example, R^{1a} can include at least one unsaturated carbon-carbon bond.

The olefin represented by formula (I) can include a C4-C16 olefin, a C4-C16 diene, a propylene oligomer (for example, propylene trimer, propylene tetramer), an isobutylene oligomer (for example, bis-isobutylene, triisobutylene), or combinations thereof.

The olefin represented by formula (I) can include 1-butene, 1-pentene, 1-hexene, 1-heptene, 1-octene, 1-nonene, 1-decene, 1-undecene (C₁₁H₂₂), 1-dodecene (C₁₂H₂₄), 1-tridecene (C₁₃H₂₆), 1-tetradecene (C₁₄H₂₈), 1-pentadecene (C₁₅H₃₀), 1-hexadecene (C₁₆H₃₂), cyclohexene, diisobutylene, trans-2-octene, norbornene, 1,5-hexadiene, 4-methyl-1-pentene, 3-methyl-1-pentene, 5-methyl-1-nonene, 3,5,5-trimethyl-1-hexene, 1,5-cyclooctadiene, 2-ethyl-1-hexene, 2-butyl-1-octene, 3,7-dimethyl-1-octene, a branched C10 monoolefin, or combinations thereof, such as 1-hexene, 1-octene, cyclohexene, diisobutylene, trans-2-octene, norbornene, 1,5-hexadiene, a branched C10 monoolefin, or combinations thereof. Isomers, such as linear, branched, acyclic, and cyclic isomers of the aforementioned olefins are contemplated.

The olefin represented by formula (I) can include a branched C10 monoolefin. Such branched C10 monoolefins can include 5-methyl-1-nonene (represented by structure I-A), 3-propyl-1-heptene (represented by structure I-B), 4-ethyl-1-octene (represented by structure I-C), 2-butyl-1-hexene (represented by structure I-D), or combinations thereof:

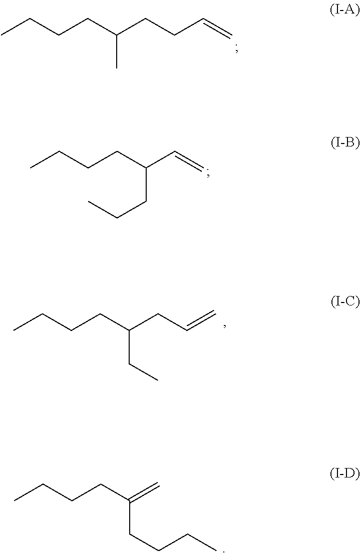

In some aspects, which can be combined with other aspects, the olefin represented by formula (I) can include 1-hexene, 1-octene, 1-decene, trans-2-octene, diisobutylene, 1,5-hexadiene, cyclohexene, norbornene, one or more branched C10 monoolefins, or combinations thereof. Additionally, or alternatively, the olefin represented by formula (I) can include one or more branched C10 monoolefins including, but not limited to, 5-methyl-1-nonene, 3-propyl-1-heptene, 4-ethyl-1-octene, 2-butyl-1-hexene, or combinations thereof.

After introducing the olefin represented by formula (I) to the reactor, the olefin represented by formula (I) can be heated, or maintained, at a temperature in a range from about 0° C. to about 200° C., such as from about 20° C. to about 180° C., such as from about 20° C. to about 165° C., such as from about 50° C. to about 150° C., such as from about 60° C. to about 140° C., such as from about 70° C. to about 130° C., such as from about 80° C. to about 120° C., such as from about 90° C. to about 110° C., such as about 100° C., or at a temperature of about 120° C. or less, such as about 100° C. or less (with a lower end of about 20° C.), or in a range from about 20° C. to about 110° C., such as from about 30° C. to about 100° C., such as from about 50° C. to about 90° C., such as from about 60° C. to about 80° C.

Additionally, or alternatively, after introducing the olefin represented by formula (I) to the reactor, the olefin represented by formula (I) can be heated, or maintained, at a temperature that is within 20° C. of a conversion temperature of the conversion conditions (described below with respect to operation 130), such as within 10° C. of the conversion temperature, such as within 5° C. of the conversion temperature. The temperature of the olefin represented by formula (I) in the reactor can be monitored by a thermocouple.

Heating, cooling, or maintaining, the temperature can be accomplished by any suitable temperature-controlling apparatus coupled to the reactor. For example, the temperature-controlling apparatus can include a fluidized temperature bath, a tube furnace, among other temperature-controlling apparatus, to heat, cool, or maintain the temperature of chemicals present inside the reactor. Alternatively, the reactor can be a jacketed reactor (also known as a double-walled reactor) in which an outer shell of the reactor is designed to control the temperature of the chemicals inside the reactor by circulating a heating fluid or cooling fluid. A tempered water system can be utilized to maintain or heat chemicals inside the reactor in a cost-effective manner. The reactor utilized in operation 110 can include any suitable reactor or vessel such as a continuous stirred tank reactor.

The olefin represented by formula (I) can be heated or maintained at the aforementioned temperature prior to introducing the mercaptan represented by formula (II), further described below with reference to operation 120.

Referring back to FIG. 1, the process 100 can further include introducing a mercaptan to the olefin represented by formula (I) to form a mixture at operation 120. The mixture formed at operation 120 includes the olefin represented by formula (I) and the mercaptan. The mercaptan can be represented by formula (II):

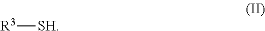

R³ of formula (II) can be a hydrogen, a substituted hydrocarbyl, or an unsubstituted hydrocarbyl. More than one mercaptan represented by formula (II) can be utilized at operation 120.

When R³ of formula (II) is a hydrocarbyl, the hydrocarbyl can have any suitable number of carbon atoms, for example, 1 to 30 carbon atoms. R³ of formula (II) can be an unsubstituted C1-C24 hydrocarbyl or a substituted C1-C24 hydrocarbyl, such as a substituted C1-C24 hydrocarbyl.

R³ of formula (II) can be, linear or branched, saturated or unsaturated, cyclic or acyclic, monocyclic or polycyclic, aromatic or non-aromatic. R³ of formula (II) can be hydrogen, a linear C1-C24 substituted hydrocarbyl, a branched C1-C24 substituted hydrocarbyl, a cyclic C1-C24 substituted hydrocarbyl, an acyclic C1-C24 substituted hydrocarbyl, or combinations thereof. Regarding saturation, R³ of formula (II) can be, independently, fully saturated, partially unsaturated, or fully unsaturated. R³ of formula (II) can include at least one carbon-carbon double bond. Monocyclic and polycyclic ring structures for R³ can be any suitable monocyclic ring structure described herein, for example, C4-C40 monocyclic or polycyclic ring structure, such as a C4-C30 monocyclic or polycyclic ring structure, etc.

R³ of formula (II) can be a C1-C20 substituted hydrocarbyl, such as a C2-C16 substituted hydrocarbyl, such as a C2-C14 substituted hydrocarbyl, such as a C2-C10 substituted hydrocarbyl, such as a C3-C8 substituted hydrocarbyl, or a C2-C7 substituted hydrocarbyl, such as a C2-C6 substituted hydrocarbyl.

R³ of formula (II) can be a C1-C20 unsubstituted hydrocarbyl, such as a C2-C16 unsubstituted hydrocarbyl, such as a C2-C14 unsubstituted hydrocarbyl, such as a C2-C10 unsubstituted hydrocarbyl, such as a C3-C8 unsubstituted hydrocarbyl, or a C2-C7 unsubstituted hydrocarbyl, such as a C2-C6 unsubstituted hydrocarbyl.

R³ of formula (II) can include a hydroxyl group, a carboxylic acid group, an ester group, an amine group, an amide group, a nitrile group, a cyano group, a silicon-containing group, or combinations thereof, such as a hydroxyl group, a carboxylic acid group, an ester group, or combinations thereof.

The mercaptan represented by formula (II) can be represented by formula (II-A):

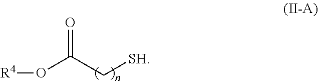

R⁴ of formula (II-A) can be hydrogen or any suitable hydrocarbyl described herein such as those described with respect to R³. In some aspects, which can be combined with other aspects, R⁴ of formula (II-A) can be hydrogen, methyl, ethyl, or propyl, such as hydrogen or methyl. “n” of formula (II-A) can be an integer such as 1, 2, or 3, such as 1 or 2.

The mercaptan represented by formula (II-A) can include thioglycolic acid (structure II-A1), 3-mercaptopropanoic acid (structure II-A2), methylthioglycolate (structure II-A3), methyl-3-mercaptopropionate (structure II-A4), or combinations thereof:

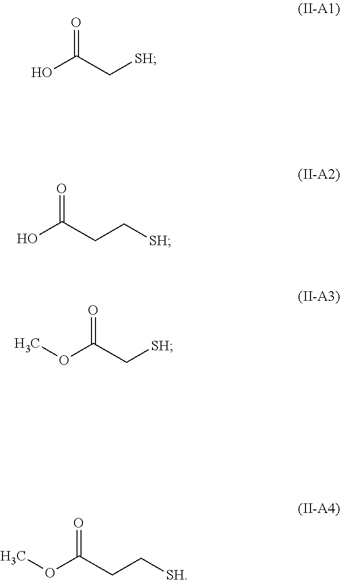

The mercaptan represented by formula (II) can include a mercaptan comprising at least one hydroxyl group (—OH). Illustrative, but non-limiting examples of mercaptans comprising at least one hydroxyl group can include beta-mercaptoethanol (structure II-B1), 3-mercaptopropanol (II-B2), 4-sulfanylbutan-1-ol (II-B3), 1-mercapto-2-propanol (II-B4), or combinations thereof:

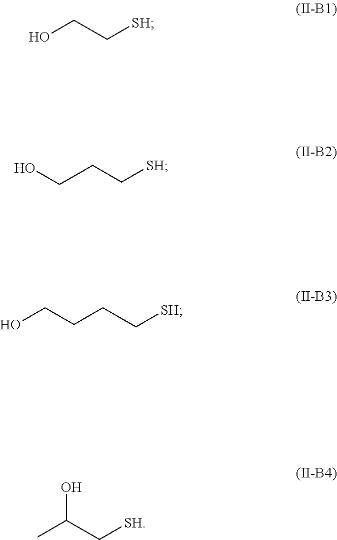

In some aspects, which can be combined with other aspects, the mercaptan represented by formula (II) can include beta-mercaptoethanol, 3-mercaptopropanol, 1-mercapto-2-propanol, 4-sulfanylbutan-1-ol, thioglycolic acid, 3-mercaptopropanoic acid, methylthioglycolate, methyl-3-mercaptopropionate, or combinations thereof.

The mercaptan of formula (II) can include amides (for example, formula (II-C1)), nitriles (for example, formula (II-C2)), anhydrides (for example, formula (II-C3)), imides (for example, formula (II-C4)) among other functional groups:

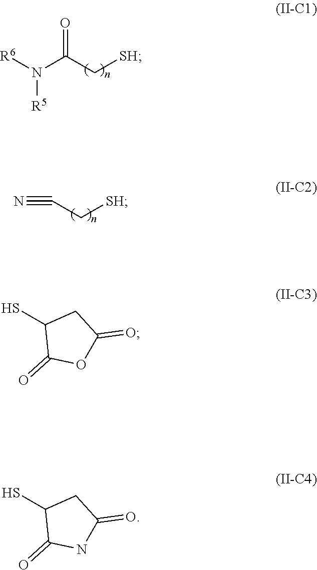

Each “n” of formula (II-C1) and formula (II-C2) can be, independently, an integer such as 1, 2, or 3, such as 1 or 2.

Referring back to operation 120, a rate of introducing (also referred to as a rate of addition) the mercaptan represented by formula (II) to the olefin represented by formula (I) can be controlled. As the reaction is exothermic, the heat of reaction can be advantageously utilized to reduce the need for the addition of external heat or energy. For example, introducing the mercaptan represented by formula (II) to the olefin represented by formula (I) can be performed at a rate such that the temperature stays near conversion temperature (for example, ±10° C. of the conversion temperature) while reducing or eliminating the need to add external heat to the reaction mixture.

The rate of addition of mercaptan represented by formula (II) to the olefin represented by formula (I) can be utilized to control or limit side reactions, to improve energy consumption, to control or limit exothermicity, or combinations thereof.

Referring back to FIG. 1, the process 100 can further include reacting the mixture, under conversion conditions, to form a conversion product effluent comprising a functionalized organic sulfide and unreacted olefin represented by formula (I) at operation 130. Through contact of the olefin represented by formula (I) in the mixture with the mercaptan represented by formula (II) in the mixture, the functionalized organic sulfide is formed. The unreacted olefin can be present when, for example, a molar excess of olefin represented by formula (I) is utilized.

Any suitable amounts of the olefin represented by formula (I) and the mercaptan represented by formula (II) can be present in the mixture that is reacted at operation 130. For example, a molar ratio of the olefin represented by formula (I) in the mixture to the mercaptan represented by formula (II) in the mixture is in a range from about 1:1 to about 10:1, such as from about 1.05:1 to about 10:1, or from about 1:1 to about 9:1, such as from about 2:1 to about 8:1, such as from about 3:1 to about 7:1, such as from about 4:1 to about 6:1, such as about 5:1, or from about 1:1 to about 5:1, such as from about 1.5:1 to about 4.5:1, such as from about 2:1 to about 4:1, such as about 3:1 (olefin:mercaptan).

Alternatively, a molar ratio of the mercaptan represented by formula (II) in the mixture to the olefin represented by formula (I) in the mixture is in a range from about 1:1 to about 10:1, such as from about 1.05:1 to about 10:1, or from about 1:1 to about 9:1, such as from about 2:1 to about 8:1, such as from about 3:1 to about 7:1, such as from about 4:1 to about 6:1, such as about 5:1, or from about 1:1 to about 5:1, such as from about 1.5:1 to about 4.5:1, such as from about 2:1 to about 4:1, such as about 3:1 (mercaptan:olefin). A mercaptan:olefin molar ratio that is about 1:1 or more in mercaptan (e.g., a 5:1 molar ratio mercaptan:olefin) can be useful when the olefin has a high boiling point or when the olefin is expensive. An olefin with a high boiling point can be more difficult to separate from the conversion product effluent than olefins having lower boiling points.

The reaction of the olefin represented by formula (I) and the mercaptan represented by formula (II) in the mixture can be performed under any suitable conversion conditions. Conversion conditions are effective to react the olefin represented by formula (I) with the mercaptan represented by formula (II). Conversion conditions can be conducted while the olefin represented by formula (I) contacts the mercaptan represented by formula (II). Conversion conditions can include various parameters and/or operations such as mixing speeds, temperatures, periods, partial pressures, etc.

Conversion conditions can include operating the reactor, for example, reactor 230, at any suitable mixing speed such as in a range from about 100 revolutions per minute (rpm) to about 1,000 rpm, such as from about 500 rpm to about 1,000 rpm.

Conversion conditions of operation 130 can include a conversion temperature. The conversion temperature can be in a range from about 20° C. to about 180° C., such as from about 20° C. to about 165° C., such as from about 50° C. to about 150° C., such as from about 60° C. to about 140° C., such as from about 70° C. to about 130° C., such as from about 80° C. to about 120° C., such as from about 90° C. to about 110° C., such as about 100° C., or at a temperature of about 120° C. or less, such as about 100° C. or less (with a lower end of about 20° C.), or in a range from about 50° C. to about 100° C., such as from about 60° C. to about 90° C., such as from about 70° C. to about 80° C., or in a range from about 20° C. to about 80° C., such as from about 30° C. to about 70° C., such as from about 40° C. to about 60° C., such as about 50° C. The conversion temperature in the reactor, for example, reactor 230, can be monitored by a thermocouple. Heating, or maintaining, the temperature can be accomplished by any suitable temperature-controlling apparatus or jacketed reactor, such as those described herein.

Conversion conditions of operation 130 can include a conversion period. The conversion period is the period after all of the mercaptan represented by formula (II) has been added to the conclusion of the reaction. Any suitable conversion period can be utilized. For example, the conversion period can be in a range from about 5 minutes to about 48 hours, such as from about 5 minutes to about 24 hours, such as from about 10 minutes to about 20 hours, such as from about 30 minutes to about 16 hours, such as from about 45 minutes to about 12 hours, such as from about 1 hour to about 8 hours, such as from about 1.5 hours to about 4 hours, such as from about 2 hours to about 3 hours.

Conversion conditions of operation 130 can include: (a) exposing the mixture comprising the olefin of formula (I) and the mercaptan of formula (II) to a first gas; (b) exposing the mixture comprising the olefin of formula (I) and the mercaptan of formula (II) to a second gas that is different from the first gas; or (c) combinations thereof. The mixture is exposed to the first gas and/or second gas in the reactor, e.g., reactor 230 of FIG. 2.

The first gas can include air, oxygen (O₂), or a combination thereof. The second gas is different from the first gas. The second gas can include a non-reactive gas such as nitrogen (N₂), argon (Ar), helium (He), krypton (Kr), xenon (Xe), or combinations thereof. Exposing the mixture to the first gas and the second gas can be performed sequentially or concurrently.

Conversion conditions of operation 130 can include exposing the mixture comprising the olefin of formula (I) and the mercaptan of formula (II) to a partial pressure of the first gas and to a partial pressure of the second gas.

A partial pressure of the first gas within the reactor can be in a range from greater than 0 psi to about 500 psi (from greater than 0 kPa to about 3,447 kPa), such as from about 1 psi to about 500 psi (from about 6.9 kPa to about 3,447 kPa), such as from about 5 psi to about 300 psi (from about 34.5 kPa to about 2,068 kPa), such as from about 10 psi to about 250 psi (from about 68.9 kPa to about 1,724 kPa), such as from about 25 psi to about 200 psi (from about 172 kPa to about 1,379 kPa), such as from about 50 psi to about 150 psi (from about 345 kPa to about 1,034 kPa), such as about 100 psi (about 689 kPa), or in a range from greater than 0 psi to about 150 psi (from greater than 0 kPa to about 1,034 kPa), such as from about 1 psi to about 100 psi (from about 6.9 kPa to about 689 kPa), such as from about 5 psi to about 75 psi (from about 34.5 kPa to about 517 kPa), such as from about 10 psi to about 50 psi (from about 68.9 kPa to about 345 kPa), such as from about 15 psi to about 35 psi (from about 103 kPa to about 241 kPa), such as about 25 psi (about 172 kPa), and a total pressure within the reactor can be in a range from greater than 0 psi to about 500 psi (from greater than 0 kPa to about 3,447 kPa), such as from about 30 psi to about 500 psi (from about 207 kPa to about 3,447 kPa), such as from about 100 psi to about 400 psi (from about 689 kPa to about 2,758 kPa), such as from about 200 psi to about 350 psi (from about 1,379 kPa to about 2,413 kPa), such as about 300 psi (about 2,068 kPa), or in a range from greater than 0 psi to about 300 psi (from greater than 0 kPa to about 2,068 kPa). The total pressure within the reactor is the sum of the partial pressure of the first gas in the reactor and the partial pressure of the second gas in the reactor. 1 psi~6.89 kPa.

Additionally, or alternatively, a partial pressure of the second gas within the reactor can be in a range from 0 psi to about 500 psi (from 0 kPa to about 3,447 kPa), such as from about 1 psi to about 500 psi (from about 6.89 kPa to about 3,447 kPa), such as from about 150 psi to about 450 psi (from about 1,034 kPa to about 3,103 kPa), such as from about 200 psi to about 350 psi (from about 1,379 to about 2,413 kPa), such as from about 250 psi to about 300 psi (from about 1,724 kPa to about 2,068 kPa), such as from about 260 psi to about 295 psi (from about 1,793 kPa to about 2,034 kPa), such as from about 275 psi to about 290 psi (from about 1,896 kPa to about 1,999 kPa), or in a range from about 150 psi to less than 300 psi (from about 1,034 kPa to less than 2,068 kPa), such as from about 150 psi to about 299 psi (from about 1,034 kPa to about 2,062 kPa), such as from about 200 psi to about 299 psi (from about 1,379 kPa to about 2,062 kPa), such as from about 225 psi to about 295 psi (from about 1,551 kPa to about 2,034 kPa), such as from about 250 psi to about 290 psi (from about 1,724 kPa to about 1,999 kPa), such as from about 265 psi to about 285 psi (from about 1,827 kPa to about 1,965 kPa), such as about 275 psi (about 1,896 kPa), and a total pressure within the reactor can be in a range from greater than 0 psi to about 500 psi (from greater than 0 kPa to about 3,447 kPa), such as from about 30 psi to about 500 psi (from about 207 kPa to about 3,447 kPa a), such as from about 100 psi to about 400 psi (from about 689 kPa to about 2,758 kPa), such as from about 200 psi to about 350 psi (from about 1,379 kPa to about 2,413 kPa), such as about 300 psi (about 2,068 kPa), or in a range from greater than 0 psi to about 300 psi (from greater than 0 kPa to about 2,068 kPa).

Partial pressures of the second gas at about 300 psi or less (about 2,068 kPa or less) can be more economical.

Conversion conditions at operation 130 can include one or more of the following:

(a) While the mixture is reacted at a conversion temperature in a range from about 60° C. to about 90° C. and a total pressure within the reactor is about 300 psi (about 2,068 kPa), the conversion conditions can include: exposing the mixture to a partial pressure of the second gas in a range from about 270 psi to about 299 psi (from about 1,862 kPa to about 2,062 kPa), such as from about 275 psi to about 295 psi (from about 1,896 kPa to about 2,034 kPa); and exposing the mixture to a partial pressure of the first gas in a range from about 1 psi to about 25 psi (from about 6.89 kPa to about 172 kPa), such as from about 5 psi to about 25 psi (from about 34.5 kPa to about 172 kPa).

(b) While the mixture is reacted at a conversion temperature in a range from about 90° C. to about 110° C. and a total pressure within the reactor is about 300 psi (about 2,068 kPa), the conversion conditions can include: exposing the mixture to a partial pressure of the second gas in a range from about 150 to about 299 psi (from about 1,034 kPa to about 2,062 kPa), such as from about 250 psi to about 299 psi (from about 1,724 kPa to about 2,062 kPa), such as from about 275 psi to about 295 psi (from about 1,896 kPa to about 2,034 kPa); and exposing the mixture to a partial pressure of the first gas in a range from about 1 psi to about 150 psi (from about 6.89 kPa to about 1,034 kPa), such as from about 1 psi to about 50 psi (from about 6.89 kPa to about 345 kPa), such as from about 5 psi to about 25 psi (from about 34.5 kPa to about 172 kPa).

(c) While the mixture is reacted at a conversion temperature in a range from about 110° C. to about 140° C. and a total pressure within the reactor is about 300 psi (about 2,068 kPa), the conversion conditions can include: exposing the mixture to a partial pressure of the second gas in a range from about 250 psi to about 299 psi (from about 1,724 kPa to about 2,062 kPa), such as from about 275 psi to about 295 psi (from about 1,896 kPa to about 2,034 kPa); and exposing the mixture to a partial pressure of the first gas in a range from about 1 psi to about 50 psi (from about 6.89 kPa to about 345 kPa), such as from about 5 psi to about 25 psi (from about 34.5 kPa to about 172 kPa).

The inventors unexpectedly found that the partial pressure of the first gas, the partial pressure of the second gas, a conversion temperature, or combinations thereof can aid in achieving high conversion of mercaptan represented by formula (II) to a functionalized organic sulfide with high product purity.

For example, larger concentrations of the anti-Markovnikov product in the conversion product effluent can be observed at lower conversion temperatures (for example, a conversion temperature of less than 150° C.). The anti-Markovnikov product, where the mercaptan adds to the least substituted carbon of the alkene (olefin), represents a desired functionalized organic sulfide. In contrast, larger concentrations of the Markovnikov addition product in the conversion product effluent can be observed at higher conversion temperatures (for example, a conversion temperature of greater than 150° C.). Markovnikov addition products, where the mercaptan of formula (II) adds to the more substituted carbon of the olefin, is a byproduct from the reaction.

As another example, at a selected temperature, lower concentrations of disulfide byproduct in the conversion product effluent can be observed when the partial pressure of the first gas is kept below about 50 psi (below about 345 kPa), such as about 25 psi (about 172 kPa) or lower (and a lower end of the partial pressure of the first gas is greater than 0 psi (greater than 0 kPa)), with the total pressure within the reactor at 300 psi or less (2,068 or less). As a non-limiting example, at a temperature of about 100° C., and a partial pressure of the first gas at about 25 psi or below (about 172 kPa or below), no detectable amounts of disulfide byproduct can be observed. Disulfide byproducts can result from reaction of two mercaptan molecules represented by formula (II). For example, 2-hydroxyethyl disulfide is a disulfide formed from reaction of two beta-mercaptoethanol molecules.

The conversion product effluent can be a composition. A “composition” can include component(s) of the composition, reaction product(s) of two or more components of the composition, a remainder balance of remaining starting component(s), or combinations thereof.

The conversion product effluent can include a functionalized organic sulfide described herein. Components that can optionally be present in the conversion product effluent can include unreacted olefin, unreacted mercaptan, byproducts from the reaction, or combinations thereof. The unreacted olefin is represented by formula (I) and the unreacted mercaptan is represented by formula (II). Byproducts from the reaction can include a Markovnikov addition product, a disulfide byproduct, or combinations thereof. Markovnikov addition products, as a byproduct, are described above. Disulfide byproducts are also described above.

Functionalized organic sulfides of the present disclosure can include any suitable reaction product from the reaction of the olefin represented by formula (I) with the mercaptan represented by formula (II). Such functionalized organic sulfides can include, for example, a thioether carboxylic acid, a thioether alcohol, a thioether ester, or combinations thereof.

Functionalized organic sulfides described herein can include a thioether carboxylic acid represented by formula (III-A), formula (III-B), formula (III-C), formula (III-D), formula (III-E1), formula (III-E2), formula (III-F), formula (III-G), formula (III-H), or combinations thereof:

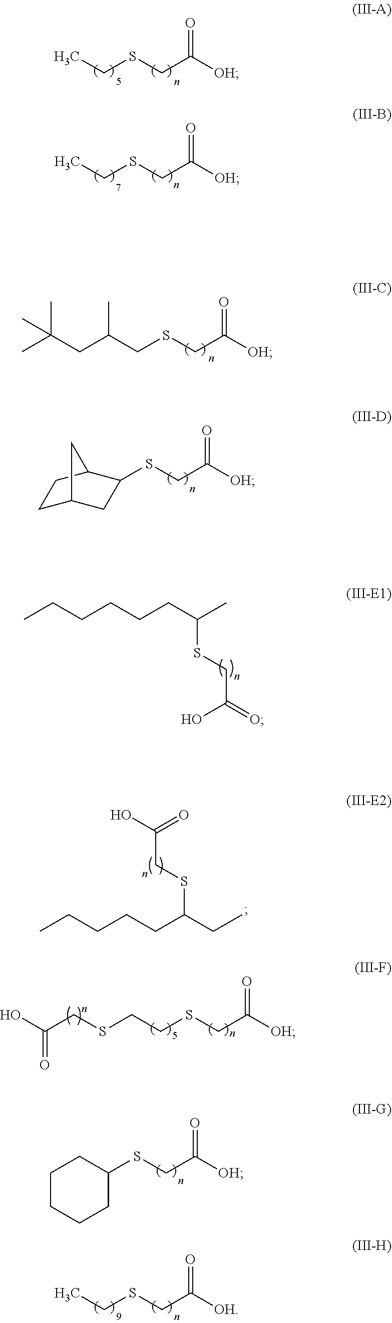

Each n of formulas (III-A)-(III-H) can be, independently, 1 or 2.

The thioether carboxylic acids represented by formulas (III-A)-(III-H) can be formed by reaction of thioglycolic acid (n=1) or 3-mercaptopropanoic acid (n=2) with: 1-hexene (formula (III-A)); 1-octene (formula (III-B)); diisobutylene (formula (III-C)); norbornene (formula (III-D)); trans-2-octene (formula (III-E1) and/or formula (III-E2)); 1,5-hexadiene (formula (III-F)); cyclohexene (formula (III-G)); or 1-decene (formula (III-H)). With respect to trans-2-octene, the reaction with either thioglycolic acid (n=1) or 3-mercaptopropanoic acid (n=2) can produce a mixture of the thioether carboxylic acids represented by formula (III-E1) and formula (III-E2).

Functionalized organic sulfides described herein can include a thioether carboxylic acid represented by formula (III-I), formula (III-J), formula (III-K), formula (III-L), formula (III-M), formula (III-N), formula (III-O), formula (III-P), or combinations thereof:

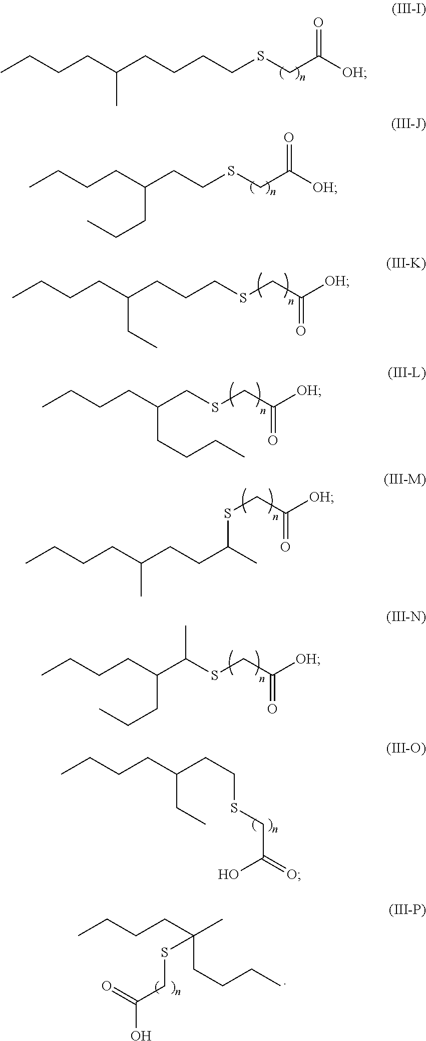

Each n of formulas (III-I)-(III-P) can be, independently, 1 or 2.

The thioether carboxylic acids represented by formulas (III-I)-(III-P) can be formed by reaction of thioglycolic acid (n=1) or 3-mercaptopropanoic acid (n=2) with a branched C10 monoolefin, such as a branched C10 monoolefin described herein.

The functionalized organic sulfide can include a thioether alcohol represented by formula (IV-A), formula (IV-B), formula (IV-C), formula (IV-D), formula (IV-E1), formula (IV-E2), formula (IV-F), formula (IV-G), formula (IV-H), formula (IV-I), formula (IV-J), isomers thereof, or combinations thereof:

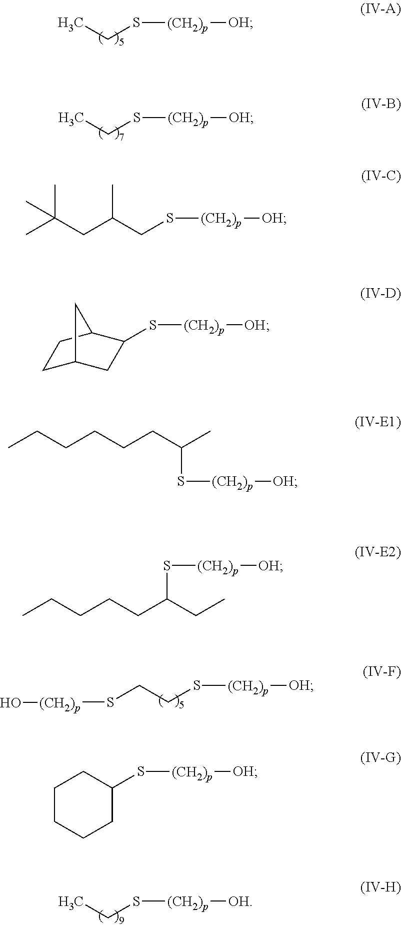

Each p of formulas (IV-A)-(IV-H) can be, independently, 2, 3, or 4.

The hydroxyl (—OH) of the —(CH₂)ₚ—OH group of formulas (IV-A)-(IV-H) can be present as a secondary alcohol. In such cases, a —CH₂— present in the —(CH₂)ₚ— group is a —CH— group and another —CH₂— present in the —(CH₂)ₚ— group is a —CH₃ group. For example, the thioether alcohols represented by formulas (IV-I) and (IV-J) can be formed by reaction of 1-mercapto-2-propanol with: 1-hexene (formula (IV-I)); or diisobutylene (formula (IV-J) as shown below.

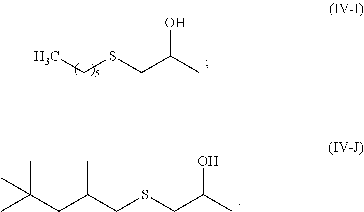

The thioether alcohols represented by formulas (IV-A)-(IV-J) can be formed by reaction of beta-mercaptoethanol (p=2), 3-mercaptopropanol (p=3), or 4-sulfanylbutan-1-ol (p=4) with: 1-hexene (formula (IV-A)); 1-octene (formula (IV-B)); diisobutylene (formula (IV-C)); norbornene (formula (IV-D)); trans-2-octene (formula (IV-E1) and/or formula (IV-E2)); 1,5-hexadiene (formula (IV-F)); cyclohexene (formula (IV-G)); or 1-decene (formula (IV-H)). With respect to trans-2-octene, the reaction with either beta-mercaptoethanol (p=2), 3-mercaptopropanol (p=3), or 4-sulfanylbutan-1-ol (p=4) can produce a mixture of the thioether alcohols represented by formula (IV-E1) and formula (IV-E2).

Functionalized organic sulfides of the present disclosure can include a thioether alcohol represented by formula (IV-K), formula (IV-L), formula (IV-M), formula (IV-N), formula (IV-0), formula (IV-P), formula (IV-Q), formula (IV-R), or combinations thereof:

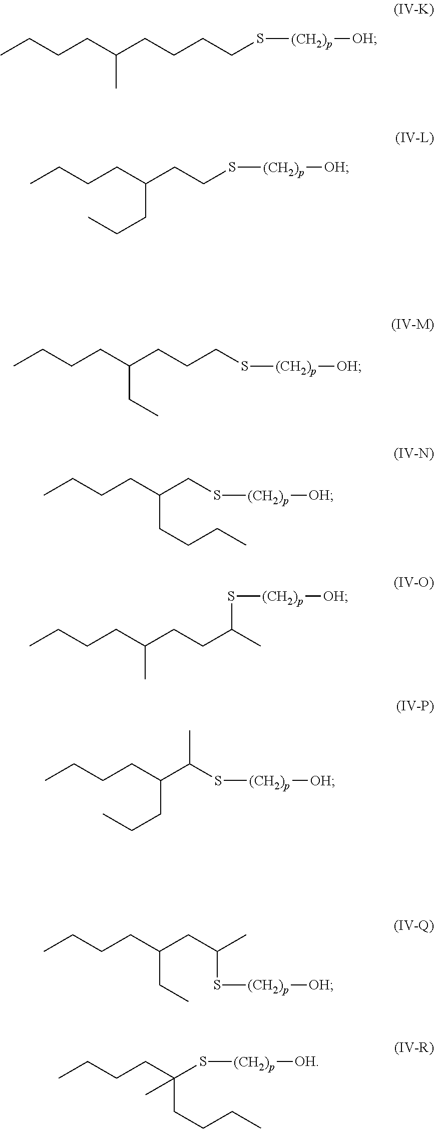

Each p of formulas (IV-K)-(IV-R) can be, independently, 2, 3, or 4.

The thioether alcohols represented by formulas (IV-K)-(IV-R) can be formed by reaction of beta-mercaptoethanol (p=2), 3-mercaptopropanol (p=3), or 4-sulfanylbutan-1-ol (p=4) with a branched C10 monoolefin, such as a branched C10 monoolefin described herein.

The hydroxyl (—OH) of the —(CH₂)ₚ—OH group of formulas (IV-K)-(IV-R) can be present as a secondary alcohol. In such cases, a —CH₂— present in the —(CH₂)ₚ— group is a —CH— group and another —CH₂— present in the —(CH₂)ₚ— group is a —CH₃ group.

Functionalized organic sulfides of the present disclosure can include a thioether ester represented by formula (V-A), formula (V-B), formula (V-C), formula (V-D), formula (V-E1), formula (V-E2), formula (V-F), formula (V-G), formula (V-H), or combinations thereof:

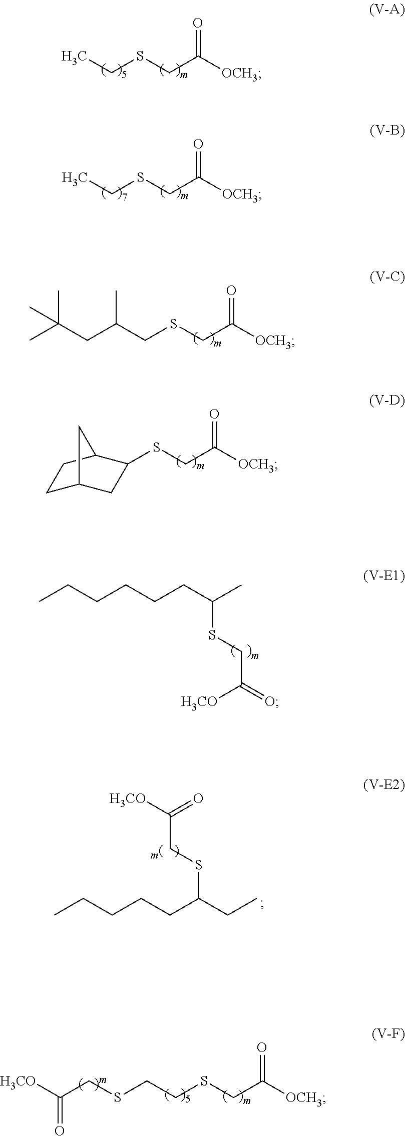

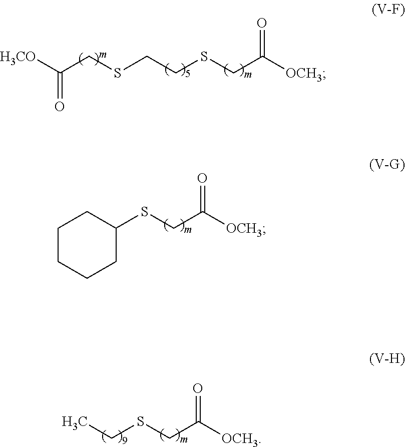

Each m of formulas (V-A)-(V-H) can be, independently, 1 or 2.

The thioether esters represented by formulas (V-A)-(V-H) can be formed by reaction of methylthioglycolate (m=1) or methyl-3-mercaptopropionate (m=2) with: 1-hexene (formula (V-A)); 1-octene (formula (V-B)); diisobutylene (formula (V-C)); norbornene (formula (V-D)); trans-2-octene (formula (V-E1) and/or formula (V-E2)); 1,5-hexadiene (formula (V-F)); cyclohexene (formula (V-G)); or 1-decene (formula (V-H)). With respect to trans-2-octene, the reaction with either methylthioglycolate (m=1) or methyl-3-mercaptopropionate (m=2) can produce a mixture of the thioether esters represented by formula (V-E1) and formula (V-E2).

Functionalized organic sulfides of the present disclosure can include a thioether ester represented by formula (V-I), formula (V-J), formula (V-K), formula (V-L), formula (V-M), formula (V-N), formula (V-O), formula (V-P), or combinations thereof:

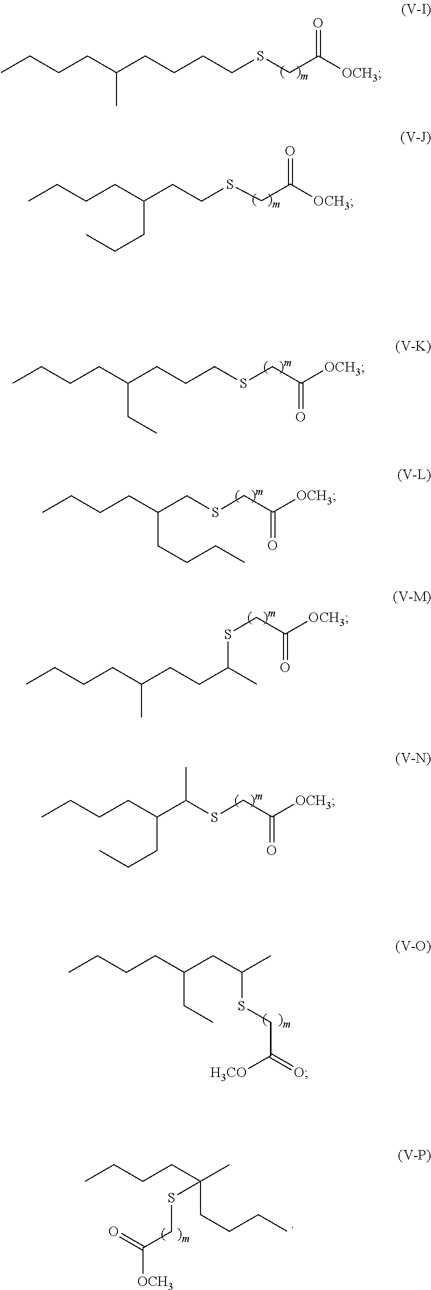

Each m of formulas (V-I)-(V-P) can be, independently, 1 or 2.

The thioether esters represented by formulas (V-I)-(V-P) can be formed by reaction of methylthioglycolate (m=1) or methyl-3-mercaptopropionate (m=2) with a branched C10 monoolefin, such as a branched C10 monoolefin described herein.

Referring back to FIG. 1, the process 100 can further include separating the unreacted olefin from the functionalized organic sulfide of the conversion product effluent at operation 140. Any suitable separation technique can be utilized, such as a liquid-liquid separation technique and/or liquid-vapor separation technique, to separate the unreacted olefin from the conversion product effluent. For example, suitable liquid-liquid separation techniques and/or liquid-vapor separation techniques can include performing a distillation, a vacuum distillation, a flash evaporation, a fractionation, an extraction, a decantation, a coalescence, or combinations thereof, on the conversion product effluent. The unreacted olefin separated can be referred to as a fraction, an effluent, or an output stream.

After separation of the unreacted olefin from the conversion product effluent, the conversion product effluent includes the functionalized organic sulfide and can further optionally include unreacted mercaptan represented by formula (II), byproducts from the reaction at operation 130, or combinations thereof. After separating the unreacted olefin from the conversion product effluent at operation 140, an amount of unreacted mercaptan in the conversion product effluent can be about 5% or less, such as about 1 wt % or less, such as about 0.5 wt % or less, such as about 0.1 wt % or less, such as below 0.1 wt % based on a total wt % of the conversion product effluent after the separation of the unreacted olefin (at operation 140), the total wt % of the conversion product effluent after the separation of the unreacted olefin is 100 wt %. Low amounts of unreacted mercaptan can be beneficial to fall within regulatory requirements. For example, various countries regulate the amount of beta-mercaptoethanol in products.

The process 100 can optionally include separating the functionalized organic sulfide from the conversion product effluent. Any suitable separation technique such as those aforementioned liquid-liquid separation techniques and/or liquid-vapor separation techniques can be utilized to separate the functionalized organic sulfide from the conversion product effluent. The functionalized organic sulfide separated can be referred to as a fraction, an effluent, or an output stream.

In some aspects, which can be combined with other aspects, one or more of the unreacted olefin and the functionalized organic sulfide can be separated from the conversion product effluent concurrently or sequentially.

The conversion product effluent can include other optional components such as unreacted mercaptan, byproducts, or combinations thereof. In such instances, the process 100 can optionally include separating unreacted mercaptan from the conversion product effluent, separating byproducts from the conversion product effluent, or combinations thereof. Both the unreacted mercaptan and the byproducts can be referred to as fractions, effluents, or output streams. Any suitable separation technique such as those aforementioned liquid-liquid separation techniques and/or liquid-vapor separation techniques can be utilized to separate the unreacted mercaptan from the conversion product effluent and to separate the byproducts from the conversion product effluent.

In some aspects, which can be combined with other aspects, the optional separation of the unreacted mercaptan from the conversion product effluent and the optional separation of the byproducts from the conversion product effluent can be performed concurrently or sequentially. In some aspects, which can be combined with other aspects, one or more of the following can be performed concurrently or sequentially, and in any suitable order: (a) the separation of the unreacted olefin from the conversion product effluent, (b) the optional separation of the functionalized organic sulfide from the conversion product effluent, (c) the optional separation of the unreacted mercaptan from the conversion product effluent, (d) the optional separation of the byproducts from the conversion product effluent, or combinations thereof.

Referring back to FIG. 1, the process 100 can further include introducing the unreacted olefin to the reactor at optional operation 150. Here, following separation of the unreacted olefin from the conversion product effluent, the unreacted olefin can be transferred to the reactor. At the reactor, the unreacted olefin can be heated and/or reacted with mercaptan of formula (II) under suitable conversion conditions, such as those described herein with respect to operation 130, to form more functionalized organic sulfide.

Prior to introducing the unreacted olefin to the reactor, at least a portion of the unreacted olefin can be purified. For example, the process 100 can optionally include removing impurities from the unreacted olefin, and then introducing the unreacted olefin of higher purity to the reactor. The unreacted olefin can be purified in a purification unit by performing any suitable separation technique such as those liquid-liquid separation techniques and/or liquid-vapor separation techniques described herein.

After optionally removing the functionalized organic sulfide from the conversion product effluent, the functionalized organic sulfide can be subjected to further purification if desired. For example, the process 100 can further include optionally removing impurities from the functionalized organic sulfide. The functionalized organic sulfide can be purified in a purification unit by performing any suitable separation technique such as those liquid-liquid separation techniques and/or liquid-vapor separation techniques described herein.

As described herein, one or more operations of process 100 can be solvent-free (performed without the use of a solvent). In some aspects, which can be combined with other aspects, one or more of operation 110, operation 120, operation 130, operation 140, or optional operation 150 can be solvent-free.

Aspects of the present disclosure also generally relate to new systems for forming functionalized organic sulfides. FIG. 2 is a generalized schematic flow diagram showing various implementations of processes described herein corresponding to operational areas or units in a system 200 for forming functionalized organic sulfides. Aspects and implementations of the system 200 can be combined with other aspects and implementations described herein, such as aspects and implementations of process 100. System 200 can be configured to perform one or more operations of process 100. System 200 can be a processing plant.

The system 200 includes an olefin feedstock unit 210 holding a first feed that includes olefin represented by formula (I). The first feed comprising olefin represented by formula (I) can exit the olefin feedstock unit 210 and flow through line 201 to enter a reactor 230. The reactor 230 can be configured to receive a first feed comprising the olefin represented by formula (I). The reactor 230 can include a first inlet coupled to line 201, the first inlet of the reactor 230 configured to receive the first feed. At the reactor 230, operation 110 can be performed, whereby the olefin represented by formula (I) is introduced into the reactor 230. The reactor 230 can be further configured to heat the first feed. In the reactor 230, the olefin represented by formula (I) can be heated or maintained at a selected temperature prior to introducing the mercaptan represented by formula (II).

The system 200 can further include a mercaptan feedstock unit 220 holding a second feed comprising mercaptan represented by formula (II). The second feed comprising mercaptan represented by formula (II) can exit the mercaptan feedstock unit 220 and flow through line 202 to enter the reactor 230. The reactor 230 can be further configured to receive a second feed comprising a mercaptan represented by formula (II). The reactor 230 can include a second inlet coupled to line 202, the second inlet of the reactor 230 configured to receive the second feed. At the reactor 230, operation 120 can be performed, whereby the mercaptan represented by formula (II) is introduced into the reactor 230 to form a mixture. The reactor can be further configured to mix the first feed and the second feed. To accomplish the mixing, the reactor 230 can include a mixer, such as an impeller, that is configured to mix the first feed and the second feed.

The reactor 230 can be further configured to receive a first output stream, the first output stream comprising unreacted olefin. The reactor 230 can include a third inlet coupled to line 205, the third inlet of the reactor 230 configured to receive the first output stream comprising unreacted olefin. As described below, the first output stream can be produced by separating the conversion product effluent at separation unit 260. The first output stream that includes the unreacted olefin can enter the reactor 230 by line 205.

The system 200 can further include a first gas tank 240 that holds a first gas comprising air, O₂, or a combination thereof. The first gas can be delivered to the reactor 230 via line 206. The system 200 can further include a second gas tank 250 that holds a second gas comprising any suitable non-reactive gas. Suitable non-reactive gases can include N₂, argon, or combinations thereof. The second gas can be delivered to the reactor 230 via line 207.

The reactor 230 can be further configured to expose the first feed that includes the olefin represented by formula (I) to the first gas, the second gas, or a combination thereof. The reactor 230 can be further configured to expose the second feed that includes the mercaptan represented by formula (II) to the first gas, the second gas, or a combination thereof. The reactor 230 can be further configured to expose the mixture that includes the first feed comprising the olefin represented by formula (I) and the second feed comprising the mercaptan represented by formula (II) to the first gas, the second gas, or a combination thereof.

The reactor 230 can be further configured to expose the mixture comprising the first feed and the second feed to a partial pressure of the first gas that includes air, O₂, or a combination thereof. The reactor 230 can be further configured to expose the mixture comprising the first feed and the second feed to a partial pressure of the second gas that includes the non-reactive gas such as N₂.

At the reactor 230, operation 130 can also be performed. For example, the reactor 230 can be further configured to react the mixture that includes the first feed—comprising the olefin represented by formula (I)—and the second feed—comprising the mercaptan represented by formula (II)—under conversion conditions to form the conversion product effluent that includes the functionalized organic sulfide. Conversion conditions to form the conversion product effluent are described herein.

The reactor 230 can be further configured to mix two or more of the first feed comprising the olefin represented by formula (I), the second feed comprising the mercaptan represented by formula (II), the first output stream comprising unreacted olefin, or combinations thereof. The reactor 230 can be further configured to react the second feed comprising the mercaptan represented by formula (II) and the first output stream comprising unreacted olefin to form more conversion product effluent that includes functionalized organic sulfide.

The reactor 230 can be further configured to discharge the conversion product effluent that includes the functionalized organic sulfide. The reactor 230 can include a first outlet coupled to line 203, the first outlet of the reactor 230 configured to discharge the conversion product effluent. The conversion product effluent can exit the reactor 230 and flow through line 203 to enter the separation unit 260. The separation unit 260 can be configured to receive the conversion product effluent. The separation unit 260 can include an inlet coupled to line 203, the inlet of the separation unit 260 configured to receive the conversion product effluent from the reactor 230. As described herein, the conversion product effluent can further include optional components such as unreacted olefin, unreacted mercaptan, byproducts from the reaction, or combinations thereof.

The separation unit 260 can be configured to separate the conversion product effluent into one or more (for example, a plurality) of output streams. The one or more output streams individually include one or more components present in the conversion product effluent. At the separation unit, operation 140 can be performed, whereby the unreacted olefin is separated from the conversion product effluent. As described above, the process 100 can optionally include separating the functionalized organic sulfide from the conversion product effluent. Optional components, such as unreacted mercaptan, byproducts, or combinations thereof can also optionally be separated from the conversion product effluent at separation unit 260. Accordingly, and in some aspects, the separation unit 260 can be: (a) configured to separate the unreacted olefin from the conversion product effluent; (b) optionally configured to separate the functionalized organic sulfide from the conversion product effluent; (c) optionally configured to separate optional unreacted mercaptan from the conversion product effluent; (d) optionally configured to separate optional byproducts from the conversion product effluent. One or more of separations (a), (b), (c), and (d) can be performed concurrently or sequentially, and in any suitable order.

To perform separation of the conversion product effluent, the separation unit 260 can be equipped with any suitable apparatus such as any suitable liquid-liquid and/or liquid-vapor separation apparatus. Suitable liquid-liquid separation apparatus and/or liquid-vapor separation apparatus can include those apparatus useful to perform a distillation, a vacuum distillation, a flash evaporation, a fractionation, an extraction, a decantation, a coalescence, or combinations thereof, on the conversion product effluent.

The separation unit 260 can be further configured to discharge one or more output streams (or effluents). The separation unit 260 can be configured to discharge a first output stream that includes unreacted olefin. The separation unit 260 can be further configured to discharge a second output stream that includes the functionalized organic sulfide. The separation unit 260 can be further configured to discharge an optional third output stream that includes optional unreacted mercaptan. The separation unit 260 can be further configured to discharge an optional fourth output stream that includes one or more optional byproducts.

The first output stream includes the unreacted olefin. The first output stream including the unreacted olefin can exit the separation unit 260 via the line 205. The separation unit 260 can include a first outlet coupled to line 205, the first outlet of the separation unit 260 configured to discharge the first output stream that includes the unreacted olefin. The line 205 is configured to transfer the first output stream that includes the unreacted olefin to the reactor 230. For example, line 205 can be configured to perform optional operation 150, whereby the unreacted olefin is introduced to the reactor 230. The line 205 can include a pump or other suitable element to assist in transferring the unreacted olefin from the separation unit 260 to the reactor 230.

The second output stream includes the functionalized organic sulfide. The second output stream that includes the functionalized organic sulfide can exit the separation unit 260 via line 204. The separation unit 260 can include a second outlet coupled to line 204, the second outlet of the separation unit 260 configured to discharge the second output stream that includes the functionalized organic sulfide.

The optional third output stream that includes optional unreacted mercaptan can exit the separation unit 260 via optional line 208. The separation unit 260 can include a third outlet coupled to optional line 208, the third outlet of the separation unit 260 configured to discharge the third output stream that includes the optional unreacted mercaptan. The optional fourth output stream that includes the optional one or more byproducts can exit the separation unit 260 via optional line 209. The separation unit 260 can include a fourth outlet coupled to optional line 209, the fourth outlet of the separation unit 260 configured to discharge the fourth output stream that includes the optional one or more byproducts.

Operation of the system 200 can be performed by the following illustrative, but non-limiting, procedure. A first feed comprising (or consisting essentially of, or consisting of) the olefin represented by formula (I) can be transferred from the olefin feedstock unit 210 to the reactor 230 via line 201. Operation 110 can then be performed to introduce the first feed comprising the olefin represented by formula (I) to the reactor 230. Inside the reactor 230, the first feed comprising the olefin represented by formula (I) can be exposed to the second gas. The first feed can be heated, or maintained, at a desired temperature with optional mixing. Operation 120 can then be performed to introduce the second feed comprising the mercaptan represented by formula (II) to the reactor 230 to form a mixture. Here, for example, the second feed comprising (or consisting essentially of, or consisting of) the mercaptan represented by formula (II) can be transferred from mercaptan feedstock unit 220 to the reactor 230 via line 202.

Operation 130 can then be performed to react the mixture, under conversion conditions, to form the conversion product effluent that includes the functionalized organic sulfide. The conversion product effluent can then be transferred from the reactor 230 to the separation unit 260 via line 203. Operation 140 can then be performed at the separation unit 260 to separate unreacted olefin from the conversion product effluent. The unreacted olefin, having been separated from the conversion product effluent can then be transferred from the separation unit 260 to the reactor 230 via line 205. Optional operation 150 can then be performed, whereby unreacted olefin can be introduced into the reactor. This recycled fraction of unreacted olefin can then react with mercaptan to form more conversion product effluent comprising functionalized organic sulfide. Functionalized organic sulfide can be discharged from the separation unit 260 via line 204. The optional unreacted mercaptan can be discharged from the separation unit 260 via optional line 208. The optional one or more byproducts can be discharged from the separation unit 260 via optional line 209.

Optionally one or more elements (for example, reactor 230, separation unit 260, line 203, line 205, etc.) described with respect to the system 200 can be coupled to a controller 270, as shown by those dashed lines coupled to the controller 270 and the element in FIG. 2. The controller 270 can be utilized to control, for example, one or more operating parameters of the one or more elements illustrated in the system 200, one or more operations of processes described herein (for example, one or more operations of process 100), or combinations thereof. The controller can be coupled to various elements to open and close valves, introduce streams (e.g., fluids, gases, solids), transfer streams (e.g., fluids, gases, solids), recycle streams (e.g., fluids, gases, solids), purge streams (e.g., fluids, gases, solids), to heat or maintain heat of equipment (e.g., reactor, separation unit, lines), etc. The controller can include a processor, memory, and support circuits. The processor can be one of any form of general purpose microprocessor, or a general purpose central processing unit (CPU), each of which can be used in an industrial setting, such as a programmable logic controller (PLC), supervisory control and data acquisition (SCADA) systems, or other suitable industrial controller.

The memory is non-transitory and can be one or more of readily available memory such as random access memory (RAM), read only memory (ROM), or any other form of digital storage, local or remote. The memory contains instructions, that when executed by the processor, can facilitate the operation of one or more elements illustrated in FIG. 2, one or more operations of process 100, or combinations thereof. The instructions in the memory are in the form of a program product such as a program that implements the method of the present disclosure. The program code of the program product can conform to any one of a number of different programming languages. Illustrative computer-readable storage media can include, but are not limited to: (i) non-writable storage media (for example, read-only memory devices within a computer such as CD-ROM disks readable by a CD-ROM drive, flash memory, ROM chips, or any type of solid-state non-volatile semiconductor memory) on which information is permanently stored; and (ii) writable storage media (for example, floppy disks within a diskette drive or hard-disk drive or any type of solid-state random-access semiconductor memory) on which alterable information is stored. Such computer-readable storage media, when carrying computer-readable instructions that direct the functions of the methods described herein, are examples of the present disclosure. The disclosure can be, for example, implemented as the program product stored on a computer-readable storage media (for example, memory) for use with a computer system (not shown). The program(s) of the program product define functions of the disclosure, described herein.

Although not shown in FIG. 2, it should be understood that suitable equipment for controlling, for example, temperature, pressure, and flow control of various feeds, effluents, and output streams can be used with the system 200. For example, heat exchangers can be used to cool or heat a liquid or a gas along one or more lines or within various units or reactors of the system 200. Pumps and motors can be utilized to control the rate of flow of the materials traveling or flowing through the lines and the operating pressures of various components of the system 200. Further, the system 200 can include valves or other release mechanisms for, e.g., purging gases or liquids from the system. Various process controls can be used. Such process controls can include probes and sensors such as pressure indicators, differential pressure cells, temperature indicators, thermocouples, temperature switches, resistance temperature detectors, solenoids, flowmeters, flow regulators and valves, gas analyzers, humidity sensors, radar sensors, ammeters, current meters, liquid level detectors, feed level probes, electrical drives, and combinations thereof.

Aspects described herein also generally relate to uses of the functionalized organic sulfides. For example, functionalized organic sulfides described herein can be used as, for example, a mining chemical collector.

Mining chemical collectors of the present disclosure can include any suitable functionalized organic sulfide described herein, such as a thioether carboxylic acid represented by formulas (III-A)-(III-P). Mining chemical collectors can be used to separate minerals during flotation processes by modifying surface properties of the minerals. Additionally, or alternatively, functionalized organic sulfides of the present disclosure can form at least a portion of a mining chemical collector composition.

Aspects of the present disclosure can be further understood by the following non-limiting examples. The following non-limiting examples are put forth so as to provide those of ordinary skill in the art with a complete disclosure and description of how to make and use aspects of the present disclosure, and are not intended to limit the scope of aspects of the present disclosure.

### EXAMPLES

Examples of functionalized organic sulfides described herein were prepared according to processes described herein and characterized according to the Test Methods.

### Materials

Olefins represented by formula (I) and used for the examples included 1-hexene, 1-octene, 1-decene, trans-2-octene, diisobutylene, 1,5-hexadiene, cyclohexene, norbornene, and a mixture of branched C10 monoolefins. The mixture of branched C10 monoolefins included at least one of 5-methyl-1-nonene, 3-propyl-1-heptene, 4-ethyl-1-octene, 2-butyl-1-hexene, or combinations thereof.

Mercaptans represented by formula (II) and used for the examples included beta-mercaptoethanol (BME), 3-mercaptopropanol, 1-mercapto-2-propanol, 4-sulfanylbutan-1-ol, thioglycolic acid, 3-mercaptopropanoic acid, methylthioglycolate, and methyl-3-mercaptopropionate.

### Test Methods

Gas chromatography-mass spectrometry (GC-MS) of samples was performed using electron impact ionization.

All nuclear magnetic resonance (NMR)—proton (¹H), carbon-13 (¹³C), and distortionless enhancement by polarization transfer (DEPT)—were performed using deuterated chloroform solvent.

### Example 1

In Example 1, various thioether alcohols, thioether carboxylic acids, and thioether esters were formed as example functionalized organic sulfides. Scheme 1-1 shows a reaction diagram for Example 1 between an olefin represented by formula (I) and a mercaptan represented by formula (II) to form a functionalized organic sulfide. Although only one R group is shown on the olefin (R^{A}), cyclic olefins, disubstituted olefins, and internal olefins were also investigated.

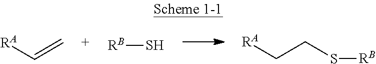

Olefin, according to Table 1, was charged to a 25 mL round-bottom flask equipped with a condenser. The olefin was used in a 5-molar excess compared to the mercaptan. The olefin was stirred under nitrogen and a mercaptan (0.92 g, 10 mmol), according to Table 1, was added slowly. The reaction mixture was stirred overnight. The reaction was expected to reach completion and form the functionalized organic sulfide when the mercaptan peak was not present or only in traces (<1%) using GC-MS analysis. Excess (unreacted) olefin was removed, followed by additional GC injections of the reaction product (conversion product effluent) containing functionalized organic sulfide.

Table 1 shows the results for the reaction of a variety of olefins represented by formula (I) with a variety of mercaptans represented by formula (II) to form functionalized organic sulfides. “Ex. No.” refers to experiment number. “Trace” and “complete” were determined by GC-MS analysis.

TABLE 1

| Ex. No. | Olefin | Mercaptan | Product |
|---|---|---|---|
| Ex. 1-1 | 1-octene | beta-mercaptoethanol | trace |
| Ex. 1-2 | cyclohexene | beta-mercaptoethanol | complete |
| Ex. 1-3 | diisobutylene | beta-mercaptoethanol | trace |
| Ex. 1-4 | trans-2-octene | beta-mercaptoethanol | complete |
| Ex. 1-5 | norbornene | beta-mercaptoethanol | complete |
| Ex. 1-6 | 1,5-hexadiene | beta-mercaptoethanol | complete |
| Ex. 1-7 | mixture of branched | beta-mercaptoethanol | trace |
| | C10 monoolefins | | |
| Ex. 1-8 | 1-hexene | thioglycolic acid | complete |
| Ex. 1-9 | 1-octene | thioglycolic acid | complete |
| Ex. 1-10 | cyclohexene | thioglycolic acid | complete |
| Ex. 1-11 | diisobutylene | thioglycolic acid | complete |
| Ex. 1-12 | trans-2-octene | thioglycolic acid | complete |
| Ex. 1-13 | norbornene | thioglycolic acid | complete |
| Ex. 1-14 | 1,5-hexadiene | thioglycolic acid | complete |
| Ex. 1-15 | mixture of branched | thioglycolic acid | complete |
| | C10 monoolefins | | |
| Ex. 1-16 | 1-hexene | 3-mercaptopropanol | complete |
| Ex. 1-17 | diisobutylene | 3-mercaptopropanol | complete |
| Ex. 1-18 | 1-hexene | 1-mercapto-2-propanol | complete |
| Ex. 1-19 | diisobutylene | 1-mercapto-2-propanol | complete |
| Ex. 1-20 | 1-hexene | methyl-3- | trace |
| | | mercaptopropionate | |
| Ex. 1-21 | diisobutylene | methyl-3- | trace |
| | | mercaptopropionate | |
| Ex. 1-22 | 1-hexene | 4-sulfanylbutan-1-ol | complete |
| Ex. 1-23 | mixture of branched | 4-sulfanylbutan-1-ol | trace |
| | C10 monoolefins | | |
| Ex. 1-24 | 1-hexene | methylthioglycolate | complete |
| Ex. 1-25 | mixture of branched | methylthioglycolate | complete |
| | C10 monoolefins | | |

Overall, the data in Table 1 demonstrates that aspects of the present disclosure can be utilized to form a variety of functionalized organic sulfides. The breadth of the reaction is excellent, advantageously forming functionalized organic sulfides from the reaction of a large scope of olefins represented by formula (I) with a large scope of mercaptans represented by formula (II). For example, linear alpha-olefins (e.g., 1-hexene and 1-octene), disubstituted olefins (e.g., diisobutylene), diolefins (e.g., 1,5-hexadiene), internal olefins (trans-2-octene), cyclic olefins (e.g., cyclohexene and norbornene), and branched olefins (mixture of branched C10 monoolefins) can be reacted with a wide range of functionalized mercaptans to form the corresponding functionalized organic sulfide. Such functionalized mercaptans represented by formula (II) included alcohol-containing mercaptans (primary and secondary alcohols), carboxylic acid-containing mercaptans, and ester-containing mercaptans.

### Example 2

In Example 2, various conversion conditions of the reaction were investigated. Scheme 2-1 shows a reaction diagram for Example 2 between an olefin represented by formula (I) and a mercaptan represented by formula (II) to form a functionalized organic sulfide. Specifically, Scheme 2-1 shows the reaction of 1-octene with BME to form 2-(octylthio)ethanol.

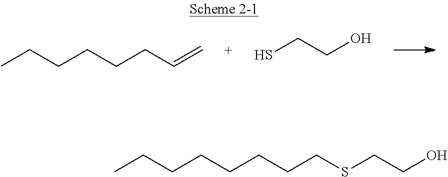

To a 500 mL 3-necked flask equipped with a condenser and a thermocouple was charged 1-octene according to Table 2. The olefin was stirred under air at the desired temperature. Beta-mercaptoethanol (BME) was added slowly to the 3-necked flask. After the addition of BME was complete, aliquots were collected after 30 minutes, 60 minutes, 120 minutes, and 16 hours (960 minutes). The aliquots were analyzed by GC-MS analysis.

Table 2 shows the results for the reaction of 1-octene with BME to form the 2-(octylthio)ethanol product as an example functionalized organic sulfide. “Rate BME added” refers to the rate at which BME was added to the 3-necked flask. “T” refers to temperature. “Conv. period” (conversion period) refers to the time at which the aliquot was collected and analyzed by GC-MS. “Area %” refers to the peak area of the individual component divided by the summed area of all of the peaks in the GC-MS chromatogram. “Purity %” refers to the area % product divided by the sum of the area % product and the area % BME.

TABLE 2

| | 1-octene/ | | | | | | | |
|---|---|---|---|---|---|---|---|---|
| | BME | | Rate | Conv. | | | | |
| | molar | | BME | period, | Area % | Area % | Area % | Purity |
| Ex. No. | ratio | T, ° C. | added | min | BME | 1-octene | product | % |
| Ex. 2-1 | 5:1 | 75 | 30 g/ | 30 | 0.801 | 82.619 | 16.58 | 95.39 |
| | | | 40 min | | | | | |
| Ex. 2-2 | 5:1 | 75 | 30 g/ | 60 | 0.651 | 83.553 | 15.797 | 96.04 |
| | | | 40 min | | | | | |
| Ex. 2-3 | 5:1 | 75 | 30 g/ | 120 | 0.439 | 81.976 | 17.586 | 97.56 |
| | | | 40 min | | | | | |
| Ex. 2-4 | 5:1 | 20 | 30 g/ | 960 | 0 | 87.766 | 12.234 | 100.00 |
| | | | 40 min | | | | | |
| Ex. 2-5 | 5:1 | 25 | 30 g/ | 30 | 0.185 | 84.183 | 15.632 | 98.83 |
| | | | 60 min | | | | | |
| Ex. 2-6 | 5:1 | 25 | 30 g/ | 60 | 0.154 | 84.234 | 15.612 | 99.02 |
| | | | 60 min | | | | | |
| Ex. 2-7 | 5:1 | 25 | 30 g/ | 120 | 0.128 | 81.415 | 18.457 | 99.31 |
| | | | 60 min | | | | | |
| Ex. 2-8 | 5:1 | 20 | 30 g/ | 960 | 0.086 | 82.765 | 17.149 | 99.50 |
| | | | 60 min | | | | | |
| Ex. 2-9 | 5:1 | 100 | 20 g/ | 30 | 0.605 | 84.613 | 14.782 | 96.07 |
| | | | 20 min | | | | | |
| Ex. 2- | 5:1 | 100 | 20 g/ | 60 | 0.389 | 84.706 | 14.905 | 97.46 |
| 10 | | | 20 min | | | | | |
| Ex. 2- | 5:1 | 100 | 20 g/ | 120 | 0.294 | 85.447 | 14.259 | 97.98 |
| 11 | | | 20 min | | | | | |
| Ex. 2- | 5:1 | 100 | 20 g/ | 960 | 0 | 84.359 | 15.641 | 100.00 |
| 12 | | | 20 min | | | | | |
| Ex. 2- | 2:1 | 100 | 30 g/ | 30 | 4.51 | 75.581 | 19.908 | 81.53 |
| 13 | | | 90 min | | | | | |
| Ex. 2- | 2:1 | 100 | 30 g/ | 60 | 3.468 | 66.406 | 30.126 | 89.68 |
| 14 | | | 90 min | | | | | |
| Ex. 2- | 2:1 | 100 | 30 g/ | 120 | 1.415 | 58.183 | 40.402 | 96.62 |
| 15 | | | 90 min | | | | | |
| Ex. 2- | 2:1 | 20 | 30 g/ | 960 | 0.201 | 78.375 | 21.424 | 99.07 |
| 16 | | | 90 min | | | | | |
| Ex. 2- | 3:1 | 100 | 30 g/ | 30 | 0.361 | 72.153 | 27.485 | 98.70 |
| 17 | | | 40 min | | | | | |
| Ex. 2- | 3:1 | 100 | 30 g/ | 60 | 0.095 | 70.268 | 29.637 | 99.68 |
| 18 | | | 40 min | | | | | |
| Ex. 2- | 3:1 | 100 | 30 g/ | 120 | 0.148 | 63.958 | 35.894 | 99.59 |
| 19 | | | 40 min | | | | | |
| Ex. 2- | 1.5:1 | 100 | 30 g/ | 30 | 2.605 | 47.35 | 50.045 | 95.05 |
| 20 | | | 30 min | | | | | |
| Ex. 2- | 1.5:1 | 100 | 30 g/ | 60 | 3.242 | 47.944 | 48.814 | 93.77 |
| 21 | | | 30 min | | | | | |
| Ex. 2- | 1.5:1 | 100 | 30 g/ | 120 | 2.605 | 47.35 | 50.045 | 95.05 |
| 22 | | | 30 min | | | | | |
| Ex. 2- | 1.5:1 | 20 | 30 g/ | 960 | 0.483 | 58.035 | 41.532 | 98.85 |
| 23 | | | 30 min | | | | | |

The data in Table 2 indicates that the solvent-free reaction can be performed at low temperatures with high conversion of the BME mercaptan and high yields of the functionalized organic sulfide. It was also observed that elevated temperatures can be utilized to overcome the non-miscibility of BME and 1-octene. The results also indicate that higher conversion temperature (Ex. 2-20) or longer conversion period (e.g., Ex. 2-4) can be utilized to bring the reaction to completion even when using a lower excess of olefin. For example, at room temperature, almost complete conversion of the BME and high product purity was observed when using a 1.5:1 molar ratio 1-octene to mercaptan (Ex. 2-23). At a temperature of 100° C., high conversion and high product purity were observed using the same molar ratio (Ex. 2-20, Ex. 2-21, and Ex. 2-22). Overall, the data in Table 2 demonstrates that aspects of the present disclosure can be utilized to form functionalized organic sulfides under various parameters.

### Example 3

In Example 3, various thioether carboxylic acids were formed as example functionalized organic sulfides. Scheme 3-1 shows a reaction diagram for Example 3 between an olefin represented by formula (I) and a mercaptan represented by formula (II) to form a thioether carboxylic acid. Although only one R group is shown on the olefin (R^{A}), cyclic olefins, disubstituted olefins, and internal olefins were also investigated.

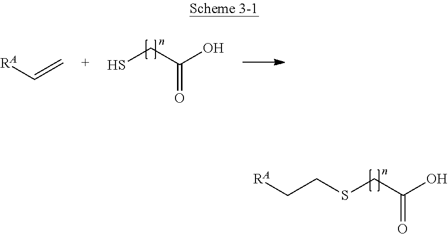

A 500 mL 3-necked flask equipped with a condenser and a thermocouple was sparged with nitrogen. Olefin was then added. The olefin was stirred under nitrogen at the desired temperature. A molar excess of the olefin was used, and specifically, a 5:1 olefin:mercaptan molar ratio was utilized. Thioglycolic acid or 3-mercaptopropanoic acid was slowly added. When the reaction was complete, a distillation column was inserted between the condenser and the 3-necked flask. Nitrogen was sparged through the reaction mixture and the temperature was slowly raised until excess (unreacted) olefin was separated. Once unreacted olefin had been completely separated, GC-MS and NMR analysis were performed on the final product.

Table 3 shows the results for the reaction to form various thioether carboxylic acids as examples of functionalized organic sulfides. The structure of products CL-1, CL-2, CL-3, CL-4, CL-5, and CL-6 are provided below the table along with ¹H and ¹³C NMR data.

TABLE 3

| | | | Temp., | |
|---|---|---|---|---|
| Ex. No. | Olefin | Mercaptan | ° C. | Product |
| Ex. 3-1 | 1-hexene | thioglycolic acid | 25 | CL-1 |
| Ex. 3-2 | cyclohexene | thioglycolic acid | 25 | CL-2 |
| Ex. 3-3 | 1-octene | thioglycolic acid | 50 | CL-3 |
| Ex. 3-4 | 1-decene | thioglycolic acid | 50 | CL-4 |
| Ex. 3-5 | mixture of branched | thioglycolic acid | 25 | CL-5 |
| | C10 monoolefins | | | |
| Ex. 3-6 | 1-hexene | 3-mercaptopropanoic | 50 | CL-6 |
| | | acid | | |

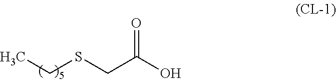

2-(hexylthio)acetic acid (CL-1): ¹H NMR (400 MHz, CDCl₃) δ 3.25 (s, 2H), 2.66 (t, J=7.4 Hz, 2H), 1.60 (p, J=7.2 Hz, 2H), 1.39 (dt, J=14.1, 6.9 Hz, 2H), 1.30 (s, 4H), 0.95-0.85 (m, 3H). ¹³C NMR (101 MHz, CDCl₃) δ 177.35, 77.55, 77.23, 76.91, 33.58, 32.88, 31.44, 28.94, 28.47, 22.59, 14.08.

Distortionless Enhancement by Polarization Transfer (DEPT) NMR was used to determine the multiplicity of various carbon atoms in CL-1. A single CH₃ signal in the DEPT spectrum was observed, validating the linear structure of the 2-(hexylthio)acetic acid obtained.

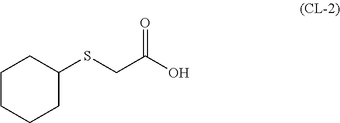 [SMILES: O=C(O)CSC1CCCCC1]

2-(cyclohexylthio)acetic acid (CL-2): ¹H NMR (400 MHz, CDCl₃) δ 3.29 (s, 2H), 2.90-2.73 (m, 1H), 2.07-1.93 (m, 2H), 1.78 (s, 2H), 1.63 (d, J=10.9 Hz, 1H), 1.40-1.17 (m, 5H). ¹³C NMR (101 MHz, CDCl₃) δ 177.33, 77.32, 77.00, 76.68, 43.84, 32.80, 31.62, 25.71, 25.54.

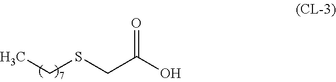

2-(octylthio)acetic acid (CL-3): ¹H NMR (400 MHz, CDCl₃) δ 3.26 (s, 1H), 2.66 (t, J=6.8 Hz, 1H), 1.61 (p, J=7.2 Hz, 2H), 1.44-1.34 (m, 2H), 1.34-1.26 (m, 7H), 0.92-0.84 (m, 3H). ¹³C NMR (101 MHz, CDCl₃) δ 177.16, 77.32, 77.00, 76.68, 33.38, 32.68, 31.68, 29.04, 29.02, 28.78, 28.62, 22.52, 13.96.

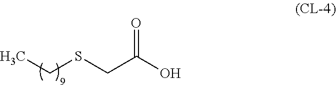

2-(decylthio)acetic acid (CL-4): ¹H NMR (400 MHz, CDCl₃) δ 3.25 (s, 2H), 2.71-2.61 (m, 2H), 1.60 (p, J=7.2 Hz, 2H), 1.44-1.33 (m, 2H), 1.26 (s, 12H), 0.94-0.80 (m, 3H). ¹³C NMR (101 MHz, CDCl₃) δ 177.15, 33.37, 32.69, 31.78, 29.44, 29.39, 29.20, 29.08, 28.79, 28.63, 22.57, 13.98.

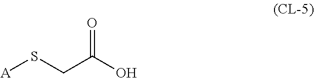

2-(mixed-decylthio)acetic acid (CL-5): “A” in structure CL-5 represents a branched C10 hydrocarbyl. ¹H NMR (400 MHz, CDCl₃) δ 3.25 (s, 2H), 2.66 (t, J=7.4 Hz, 2H), 1.67-1.50 (m, 2H), 1.26 (s, 13H), 1.10 (d, J=47.5 Hz, 2H), 0.79 (s, 7H). ¹³C NMR (101 MHz, CDCl₃) δ 177.41, 77.55, 77.23, 76.91, 38.69, 36.82, 36.75, 36.69, 35.83, 33.69, 33.63, 33.44, 33.11, 33.03, 32.97, 32.94, 32.92, 32.89, 32.77, 32.45, 32.04, 31.94, 30.59, 29.69, 29.64, 29.44, 29.38, 29.33, 29.29, 29.28, 29.04, 28.87, 26.36, 26.33, 26.29, 25.93, 23.25, 23.20, 23.16, 22.82, 22.78, 19.82, 19.76, 14.56, 14.29, 14.24, 14.22, 10.94.

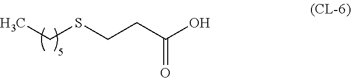

3-(hexylthio)propanoic acid (CL-6): ¹H NMR (400 MHz, CDCl₃) δ 2.85-2.74 (m, 2H), 2.71-2.62 (m, 2H), 2.58-2.48 (m, 2H), 1.58 (p, J=7.3 Hz, 2H), 1.44-1.33 (m, 2H), 1.23 (s, 4H), 0.89 (t, J=6.9 Hz, 3H). ¹³C NMR (101 MHz, CDCl₃) δ 178.35, 34.69, 32.04, 31.26, 29.34, 28.38, 26.43, 22.39, 13.87.

In all cases, the desired product was obtained. For Ex. 3-3 and Ex. 3-4, complete solidification of the reaction mixture was observed at 25° C. which prompted the use of a higher reaction temperature of 50° C. When using 3-mercaptopropanoic acid (Ex. 3-6) instead of thioglycolic acid, the reaction progressed more slowly at 25° C. Therefore, a higher reaction temperature of 50° C. was utilized.

The results demonstrated that the reaction between a carboxylic-acid containing mercaptan (thioglycolic acid or 3-mercaptopropanoic acid) and a variety of olefins occurs through anti-Markovnikov addition of the mercaptan adding to the less substituted carbon of the olefin. Overall, Example 3 shows that aspects of the present disclosure can be utilized to form a variety of thioether carboxylic acids, as examples of functionalized organic sulfides, at low temperatures.

### Example 4

In Example 4, various conversion conditions of the reaction were investigated. Scheme 4-1 shows a reaction diagram for Example 4 between an olefin represented by formula (I) and a mercaptan represented by formula (II) to form a functionalized organic sulfide. Specifically, Scheme 4-1 shows the reaction of 1-octene with BME to form 2-(octylthio)ethanol.

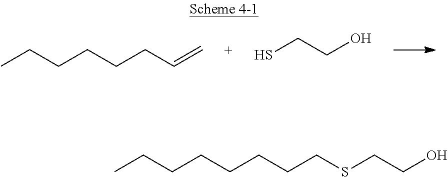

1-octene and then BME were charged to a 100 mL autoclave equipped with a stirrer and a thermocouple. The reaction mixture was stirred at the desired temperature under various partial pressures of air and various partial pressures of nitrogen. Samples were collected and analyzed by GC-MS analysis.

Table 4 shows various parameters, conversion temperature (T), partial pressure of air (P_{Air}), and partial pressure of N₂ (P_{N2}) and total pressure, as well as the results for the reaction to form 2-(octylthio)ethanol as an example functionalized organic sulfide. A 1.5:1 olefin:BME molar ratio was utilized for all of Examples (Exs.) 4-1 to 4-15, while a 1:1 olefin:BME molar ratio was utilized for Ex. 4-16. Byproducts from the reaction included diHEDS and octan-2-ylthioethanol. “diHEDS” refers to 2-hydroxyethyl disulfide, which is the disulfide product from reaction of two BME. Octan-2-ylthioethanol is the Markovnikov product (branched addition product) having the structure:

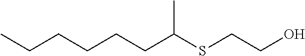

“Prod.” refers to 2-(octylthio)ethanol. “Area %” refers to the peak area of the individual component divided by the summed area of all of the peaks in the GC-MS chromatogram. Total pressure (Total P) refers to the sum of the partial pressure of air and the partial pressure of nitrogen in the reactor. Asterisks indicate no reaction. 1 psi~6.89 kPa.

TABLE 4

| | | | | | Area % | | | | |
|---|---|---|---|---|---|---|---|---|---|
| | Reaction parameters | | | | | | | Octan- | |
| | | P_{Air}, | P_{N2}, | Total P, | | 1- | | 2- | |
| Ex. No. | T, ° C. | psi | psi | psi | BME | Octene | diHEDS | ylthioethanol | Prod. |
| Ex. 4-1 | 100 | 0 | 500 | 500 | * | * | 0 | 0 | 0 |
| Ex. 4-2 | 120 | 0 | 500 | 500 | * | * | 0 | 0 | 0 |
| Ex. 4-3 | 100 | 150 | 0 | 150 | 4.63 | 23.95 | 0.00 | 4.53 | 66.89 |
| Ex. 4-4 | 100 | 300 | 0 | 300 | 0.00 | 17.97 | 2.14 | 3.39 | 64.57 |
| Ex. 4-5 | 100 | 150 | 150 | 300 | 1.10 | 20.40 | 3.32 | 3.69 | 72.09 |
| Ex. 4-6 | 100 | 100 | 200 | 300 | 0.00 | 18.97 | 1.06 | 3.27 | 77.30 |
| Ex. 4-7 | 100 | 50 | 250 | 300 | 0.65 | 19.02 | 0.55 | 3.00 | 76.03 |
| Ex. 4-8 | 100 | 25 | 275 | 300 | 0.92 | 22.41 | 0.00 | 2.47 | 70.68 |
| Ex. 4-9 | 100 | 10 | 200 | 300 | 0.00 | 14.30 | 0.00 | 7.53 | 72.29 |
| Ex. 4-10 | 100 | 5 | 295 | 300 | 7.67 | 29.81 | 0.00 | 1.47 | 55.92 |
| Ex. 4-11 | 125 | 5 | 295 | 300 | 9.59 | 26.49 | 0.74 | 4.03 | 63.19 |
| Ex. 4-12 | 150 | 5 | 295 | 300 | 7.19 | 18.91 | 0.69 | 36.77 | 29.56 |
| Ex. 4-13 | 125 | 10 | 290 | 300 | 9.73 | 28.36 | 0.00 | 9.09 | 51.23 |
| Ex. 4-14 | 150 | 10 | 290 | 300 | 6.79 | 25.00 | 0.44 | 2.98 | 65.71 |
| Ex. 4-15 | 75 | 10 | 290 | 300 | 8.69 | 27.28 | 0.00 | 2.30 | 60.68 |
| Ex. 4-16 | 100 | 100 | 200 | 300 | 0.00 | 8.65 | 2.93 | 5.11 | 84.20 |

The data in Table 4 shows the unexpected effect of air partial pressure, nitrogen partial pressure, and conversion temperature on product purity. Larger concentrations of byproducts in the samples (a conversion product effluent) analyzed by GC-MS were observed at higher temperature. For example, larger concentrations of the branched addition product, octan-2-ylthioethanol, were observed at higher conversion temperatures of 150° C. (Ex. 4-12). Though, this effect appeared to diminish at higher air partial pressures (Ex. 4-14).

In addition, at a selected temperature, lower concentrations of byproducts in the samples were observed at lower air partial pressures. For example, at 100° C., lower concentrations of diHEDS disulfide byproduct were observed as the air partial pressure decreased from 150 psi (Ex. 4-5: Area % of 3.32) to 50 psi (Ex. 4-7: Area % of 0.55), and the diHEDS disulfide byproduct was not detectable at air partial pressures of 25 psi and below 25 psi (Ex. 4-8, Ex. 4-9, and Ex. 4-10). The results also demonstrate that complete BME conversion can be accomplished while using a 1:1 molar ratio of 1-octene to BME (Ex. 4-16).

### Example 5: Potential Mechanism

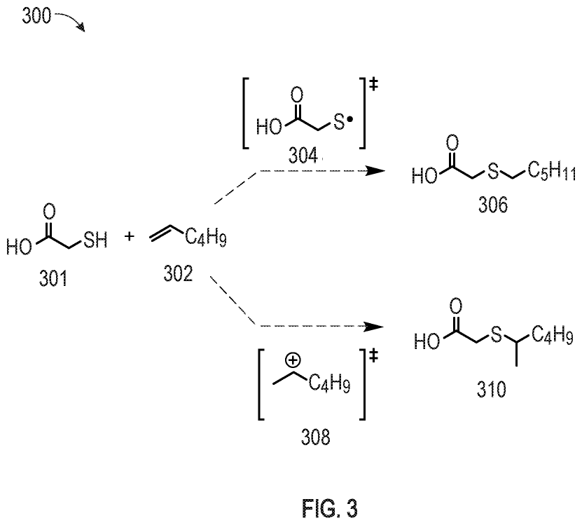

Theoretically, reaction of an olefin represented by formula (I) with a mercaptan represented by formula (II) can form linear addition products or branched addition products as a result of anti-Markovnikov addition or Markovnikov addition, respectively. As a non-limiting illustration, FIG. 3 shows a potential mechanism 300 for the reaction between thioglycolic acid 301 and 1-hexene 302. As shown, the reaction can proceed through a radical mechanism via intermediate thiyl radical 304 to obtain the linear addition product 306 (CL-1, 2-(hexylthio)acetic acid). Additionally, or alternatively, the reaction can proceed through an acid-catalyzed olefin activation via intermediate 308 to obtain the branched addition product 310. DEPT NMR and GC-MS was performed on the product and indicated that the product was mostly the linear addition product, suggesting that the radical mechanism dominates. Only trace amounts of the branched addition product 310 were observed by GC-MS under the conditions investigated.

The results provided herein demonstrate an uncatalyzed reaction pathway for forming functionalized organic sulfides. While not wishing to be bound by any theory, it is believed that O₂ can catalyze the reaction between the olefin represented by formula (I) and the mercaptan represented by formula (II) as described herein.

Aspects described herein generally relate to new processes for forming functionalized organic sulfides, to new systems for forming functionalized organic sulfides, and to uses of the functionalized organic sulfides. Aspects of the present disclosure enable formation of functionalized organic sulfides from a wide variety of olefin and mercaptan starting materials and without the use of solvents. Formation of functionalized organic sulfides by aspects described herein is compatible with various functional groups including alcohols, esters, and carboxylic acids. Advantageously, functionalized organic sulfides can be formed without the addition of catalyst or an energy source such as light. When heat is utilized, functionalized organic sulfides can be formed at temperatures lower than conventional methods.

### Aspects of the Disclosure

The present disclosure provides, among others, the following aspects, each of which can be considered as optionally including any alternate aspects:

Aspect 1. A process for forming a functionalized organic sulfide, the process comprising:
introducing, to a reactor, an olefin represented by formula (I):

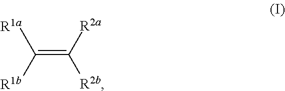

wherein: each of R^{1a}, R^{1b}, R^{2a}, and R^{2b} of formula (I) is, independently, hydrogen, a C1-C20 unsubstituted hydrocarbyl, a C1-C20 substituted hydrocarbyl, or two or more of R^{1a}, R^{1b}, R^{2a}, or R^{2b} join together to form a C4-C30 monocyclic or polycyclic ring structure;
introducing a mercaptan to the olefin represented by formula (I) to form a mixture, the mercaptan represented by formula (II):

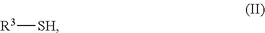

wherein R³ of formula (II) is a hydrogen, a C1-C24 substituted hydrocarbyl, or a C1-C24 unsubstituted hydrocarbyl;
reacting the mixture, under conversion conditions, to form a conversion product effluent comprising a functionalized organic sulfide and unreacted olefin represented by formula (I); and
separating the unreacted olefin from the conversion product effluent.

Aspect 2. The process according to Aspect 1, wherein the process is solvent-free, or at least the reacting the mixture is solvent-free.

Aspect 3. The process according to any one of the preceding Aspects, wherein, after separating the unreacted olefin from the conversion product effluent, the process further comprises: introducing the unreacted olefin to the reactor to react with the mercaptan represented by formula (II).

Aspect 4. The process according to any one of the preceding Aspects, wherein separating the unreacted olefin from the conversion product effluent comprises: performing any suitable liquid-liquid separation apparatus and/or liquid-vapor separation apparatus (for example, distillation, vacuum distillation, flash evaporation, fractionation, extraction, decantation, coalescence, or combinations thereof).

Aspect 5. The process according to any one of the preceding Aspects, wherein the conversion conditions comprise exposing the mixture to a first gas comprising air, O₂, or a combination thereof, a second gas comprising a non-reactive gas (for example, N₂), or a combination of the first gas and the second gas.

Aspect 6. The process according to any one of the preceding Aspects, wherein the conversion conditions comprise: exposing the mixture to a first gas comprising air, O₂, or a combination thereof, and exposing the mixture to a second gas comprising a non-reactive gas (for example, N₂).

Aspect 7. The process according to Aspect 6, wherein: a partial pressure of the first gas within the reactor is in a range from greater than 0 psi to about 500 psi, such as from about 1 psi to about 500 psi, such as from about 5 psi to about 300 psi, such as from about 10 psi to about 250 psi, such as from about 25 psi to about 200 psi, such as from about 50 psi to about 150 psi, such as about 100 psi, or in a range from greater than 0 psi to about 150 psi, such as from about 1 psi to about 100 psi, such as from about 5 psi to about 75 psi, such as from about 10 psi to about 50 psi, such as from about 15 psi to about 35 psi, such as about 25 psi; and a total pressure within the reactor is in a range from greater than 0 psi to about 500 psi, such as from about 30 psi to about 500 psi, such as from about 100 psi to about 400 psi, such as from about 200 psi to about 350 psi, such as about 300 psi, or in a range from greater than 0 psi to about 300 psi (wherein: 1 psi~6.89 kPa).

Aspect 8. The process according to any one of Aspects 6-7, wherein: a partial pressure of the second gas within the reactor is in a range from 0 psi to about 500 psi, such as from about 1 psi to about 500 psi, such as from about 150 psi to about 450 psi, such as from about 200 psi to about 350 psi, such as from about 250 psi to about 300 psi, such as from about 260 psi to about 295 psi, such as from about 275 psi to about 290 psi, or in a range from about 150 psi to less than 300 psi, such as from about 150 psi to about 299 psi, such as from about 200 psi to about 299 psi, such as from about 225 psi to about 295 psi, such as from about 250 psi to about 290 psi, such as from about 265 psi to about 285 psi, such as about 275 psi; and a total pressure within the reactor is in a range from greater than 0 psi to about 500 psi, such as from about 30 psi to about 500 psi, such as from about 100 psi to about 400 psi, such as from about 200 psi to about 350 psi, such as about 300 psi, or in a range from greater than 0 psi to about 300 psi (wherein: 1 psi~6.89 kPa).

Aspect 9. The process according to any one of the preceding Aspects, wherein the conversion conditions comprise: a conversion temperature in a range from about 20° C. to about 180° C., such as from about 20° C. to about 165° C., such as from about 50° C. to about 150° C., such as from about 60° C. to about 140° C., such as from about 70° C. to about 130° C., such as from about 80° C. to about 120° C., such as from about 90° C. to about 110° C., such as about 100° C., or in a range from about 20° C. to about 110° C., such as from about 30° C. to about 100° C., such as from about 50° C. to about 90° C., such as from about 60° C. to about 80° C., or in a range from about 50° C. to about 100° C., such as from about 60° C. to about 90° C., such as from about 70° C. to about 80° C.

Aspect 10. The process according to any one of the preceding Aspects, wherein the conversion conditions comprise: a conversion period in a range from about 5 minutes to about 24 hours, such as from about 10 minutes to about 20 hours, such as from about 30 minutes to about 16 hours, such as from about 45 minutes to about 12 hours, such as from about 1 hour to about 8 hours, such as from about 1.5 hours to about 4 hours, such as from about 2 hours to about 3 hours.

Aspect 11. The process according to any one of the preceding Aspects, wherein, prior to introducing the mercaptan represented by formula (II) to the olefin represented by formula (I) to form the mixture, the process further comprises:
heating, or maintaining, the olefin represented by formula (I) at a temperature in a range from about 20° C. to about 180° C., such as from about 20° C. to about 165° C., such as from about 50° C. to about 150° C., such as from about 60° C. to about 140° C., such as from about 70° C. to about 130° C., such as from about 80° C. to about 120° C., such as from about 90° C. to about 110° C., such as about 100° C.; or heating the olefin represented by formula (I) at a temperature in a range from about 50° C. to about 100° C., such as from about 60° C. to about 90° C., such as from about 70° C. to about 80° C., or heating, or maintaining, the olefin represented by formula (I) at a temperature that is within 20° C. of a conversion temperature of the conversion conditions, such as within 10° C. of the conversion temperature, such as within 5° C. of the conversion temperature; or combinations thereof.

Aspect 12. The process according to any one of the preceding Aspects, wherein a molar ratio of the olefin represented by formula (I) in the mixture to the mercaptan represented by formula (II) in the mixture is in a range from about 1:1 to about 10:1, such as from about 1.05:1 to about 10:1, or from about 1:1 to about 9:1, such as from about 2:1 to about 8:1, such as from about 3:1 to about 7:1, such as from about 4:1 to about 6:1, such as about 5:1, or from about 1:1 to about 5:1, such as from about 1.5:1 to about 4.5:1, such as from about 2:1 to about 4:1, such as about 3:1 (olefin:mercaptan).

Aspect 13. The process according to any one of the preceding Aspects, wherein, after separating the unreacted olefin from the conversion product effluent, an amount of unreacted mercaptan in the conversion product effluent is about 5% or less, such as about 1 wt % or less, such as about 0.5 wt % or less, such as about 0.1 wt % or less, such as below 0.1 wt % based on a total wt % of the conversion product effluent after separating the unreacted olefin, the total wt % of the conversion product effluent after separating the unreacted olefin is 100 wt %.

Aspect 14. The process according to Aspect 13, wherein the unreacted mercaptan comprises beta-mercaptoethanol.

Aspect 15. The process according to any one of the preceding Aspects, wherein: each of R^{1a} and R^{1b} of formula (I) is, independently, hydrogen, or a linear or branched, cyclic or acyclic C2-C16 unsubstituted hydrocarbyl.

Aspect 16. The process according to any one of the preceding Aspects, wherein: each of R^{2a} and R^{2b} of formula (I) is, independently, hydrogen, methyl, or ethyl.

Aspect 17. The process according to any one of the preceding Aspects, wherein: each of R^{1a} and R^{2a} of formula (I) join together to form a C4-C30 monocyclic or polycyclic ring structure.

Aspect 18. The process according to any one of the preceding Aspects, wherein: at least one of R^{1a}, R^{1b}, R^{2a}, or R^{2b} of formula (I) contains at least one unsaturated carbon-carbon bond (for example, R^{1a})

Aspect 19. The process according to any one of the preceding Aspects, wherein: at least one of R^{1a}, R^{1b}, R^{2a}, or R^{2b} of formula (I) is a C4-C16 unsubstituted hydrocarbyl, such as a C5-C12 unsubstituted hydrocarbyl, such as a C6-C10 unsubstituted hydrocarbyl.

Aspect 20. The process according to any one of the preceding Aspects, wherein the olefin represented by formula (I) comprises a C4-C16 olefin, a propylene oligomer, an isobutylene oligomer, a C4-C16 diene, or combinations thereof.

Aspect 21. The process according to any one of the preceding Aspects, wherein the olefin represented by formula (I) comprises 1-butene, 1-pentene, 1-hexene, 1-heptene, 1-octene, 1-nonene, 1-decene, 1-undecene (C11H₂₂), 1-dodecene (C12H₂₄), 1-tridecene (C13H₂₆), 1-tetradecene (C14H₂₈), 1-pentadecene (Cl₅H₃₀), 1-hexadecene (C16H₃₂), cyclohexene, diisobutylene, trans-2-octene, norbornene, 1,5-hexadiene, 4-methyl-1-pentene, 3-methyl-1-pentene, 5-methyl-1-nonene, 3,5,5-trimethyl-1-hexene, 1,5-cyclooctadiene, 2-ethyl-1-hexene, 2-butyl-1-octene, 3,7-dimethyl-1-octene, a branched C10 monoolefin, or combinations thereof, such as 1-hexene, 1-octene, cyclohexene, diisobutylene, trans-2-octene, norbornene, 1,5-hexadiene, a branched C10 monoolefin, or combinations thereof.

Aspect 22. The process according to Aspect 21, wherein the branched C10 monoolefin comprises 5-methyl-1-nonene (represented by structure I-A), 3-propyl-1-heptene (represented by structure I-B), 4-ethyl-1-octene (represented by structure I-C), 2-butyl-1-hexene (represented by structure I-D), or combinations thereof:

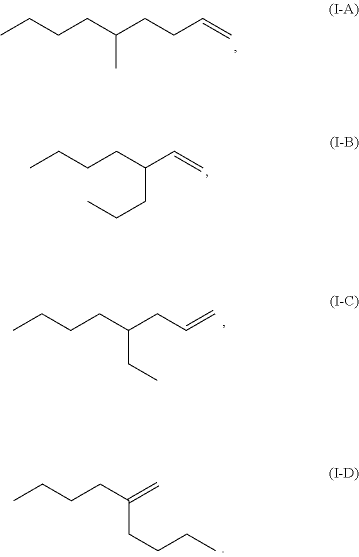

Aspect 23. The process according to any one of the preceding Aspects, wherein: R³ of formula (II) is a linear or branched, cyclic or acyclic C1-C24 substituted hydrocarbyl, such as a linear or branched C1-C20 substituted hydrocarbyl.

Aspect 24. The process according to Aspect 23, wherein: R³ of formula (II) is a linear or branched C2-C14 substituted hydrocarbyl, such as a linear or branched C2-C10 substituted hydrocarbyl, such as a linear or branched C3-C8 substituted hydrocarbyl, or a linear or branched C2-C7 substituted hydrocarbyl, such as a linear or branched C2-C6 substituted hydrocarbyl.

Aspect 25. The process according to any one of the preceding Aspects, wherein: R³ of formula (II) comprises a hydroxyl group, a carboxylic acid group, an ester group, an amine group, an amide group, a nitrile group, a cyano group, or a silicon-containing group, such as a hydroxyl group, a carboxylic acid group, or an ester group.

Aspect 26. The process according to any one of the preceding Aspects, wherein the mercaptan represented by formula (II) is a mercaptan represented by formula (II-A):

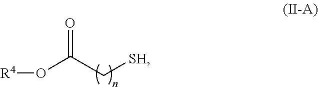

wherein: n of formula (II-A) is an integer from 1-3, such as 1 or 2; and R⁴ of formula (II-A) is hydrogen, methyl, or ethyl.

Aspect 27. The process according to Aspect 26, wherein the mercaptan represented by formula (II-A) comprises thioglycolic acid (structure II-A1), 3-mercaptopropanoic acid (structure II-A2), methylthioglycolate (structure II-A3), methyl-3-mercaptopropionate (structure II-A4), or combinations thereof.

Aspect 28. The process according to any one of the preceding Aspects, wherein the mercaptan represented by formula (II) is a mercaptan comprising a hydroxyl group.

Aspect 29. The process according to Aspect 28, wherein the mercaptan comprising the hydroxyl group comprises beta-mercaptoethanol (structure II-B1), 3-mercaptopropanol (II-B2), 4-sulfanylbutan-1-ol (II-B3), 1-mercapto-2-propanol (II-B4), or combinations thereof.

Aspect 30. The process according to any one of the preceding Aspects, wherein the functionalized organic sulfide comprises a thioether carboxylic acid, a thioether alcohol, a thioether ester, or combinations thereof.

Aspect 31. The process according to Aspect 30, wherein the thioether carboxylic acid is represented by formula (III-A), formula (III-B), formula (III-C), formula (III-D), formula (III-E1), formula (III-E2), formula (III-F), formula (III-G), formula (III-H), or combinations thereof, wherein

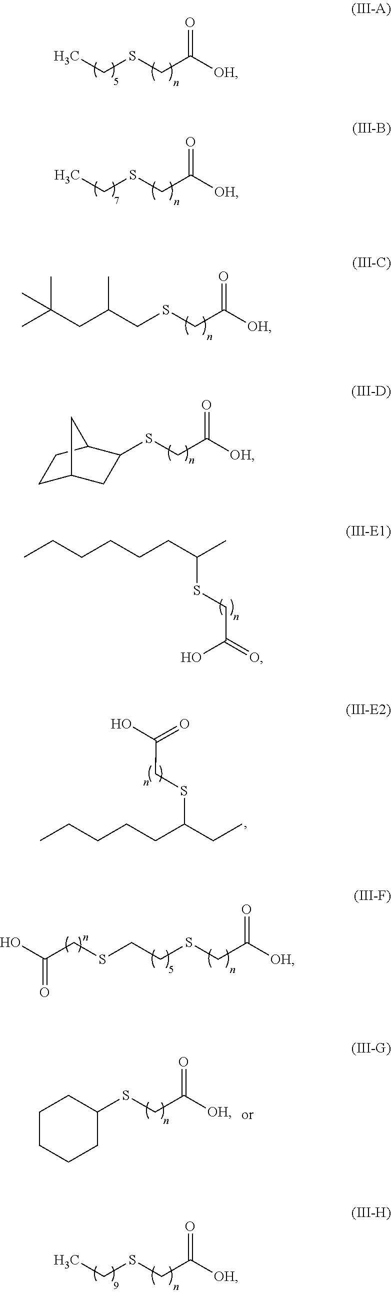

wherein each n of formulas (III-A)-(III-H) is, independently, 1 or 2.

Aspect 32. The process according to Aspect 30, wherein the thioether carboxylic acid is represented by formula (III-I), formula (III-J), formula (III-K), formula (III-L), formula (III-M), formula (III-N), formula (III-O), formula (III-P), or combinations thereof:

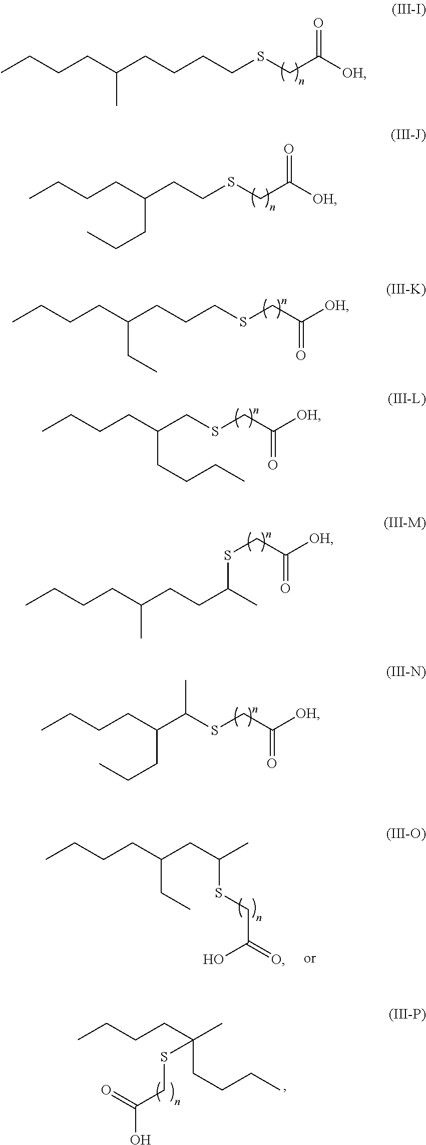

wherein each n of formulas (III-I)-(III-P) is, independently, 1 or 2.

Aspect 33. The process according to Aspect 30, wherein the thioether alcohol is represented by represented by formula (IV-A), formula (IV-B), formula (IV-C), formula (IV-D), formula (IV-E1), formula (IV-E2), formula (IV-F), formula (IV-G), formula (IV-H), formula (IV-I), formula (IV-J), or combinations thereof:

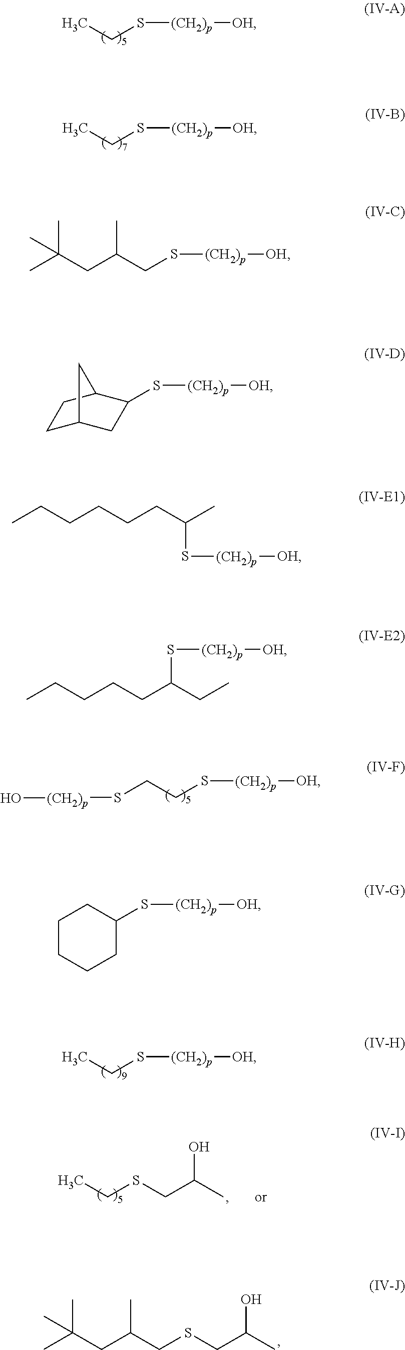

wherein each p of formulas (IV-A)-(IV-H) is, independently, 2, 3, or 4.

Aspect 34. The process according to Aspect 30, wherein the thioether alcohol is represented by formula (IV-K), formula (IV-L), formula (IV-M), formula (IV-N), formula (IV-0), formula (IV-P), formula (IV-Q), formula (IV-R), or combinations thereof:

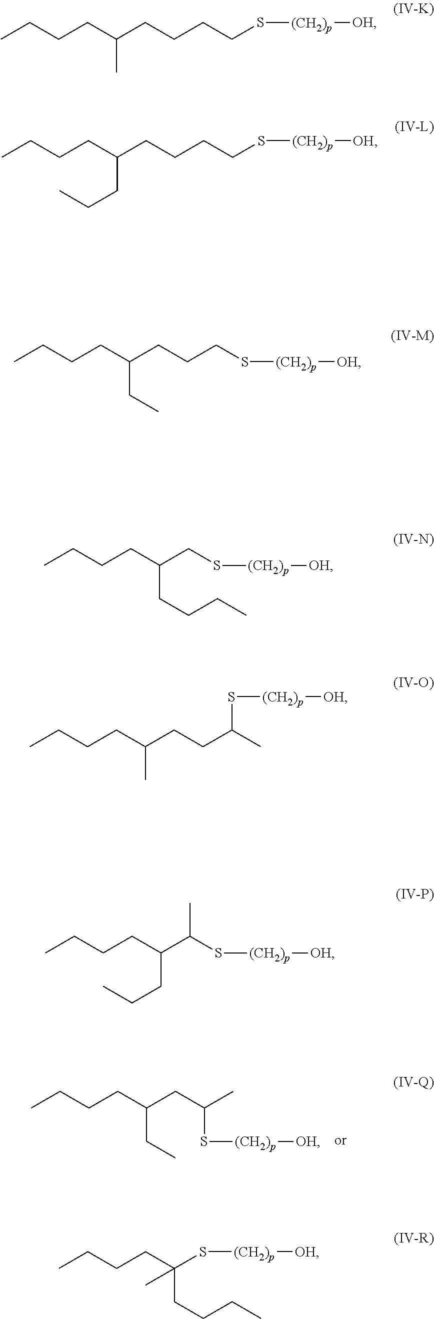

wherein each p of formulas (IV-K)-(IV-R) is, independently, 2, 3, or 4.

Aspect 35. The process according to Aspect 30, wherein the thioether ester is represented by formula (V-A), formula (V-B), formula (V-C), formula (V-D), formula (V-E1), formula (V-E2), formula (V-F), formula (V-G), formula (V-H), or combinations thereof:

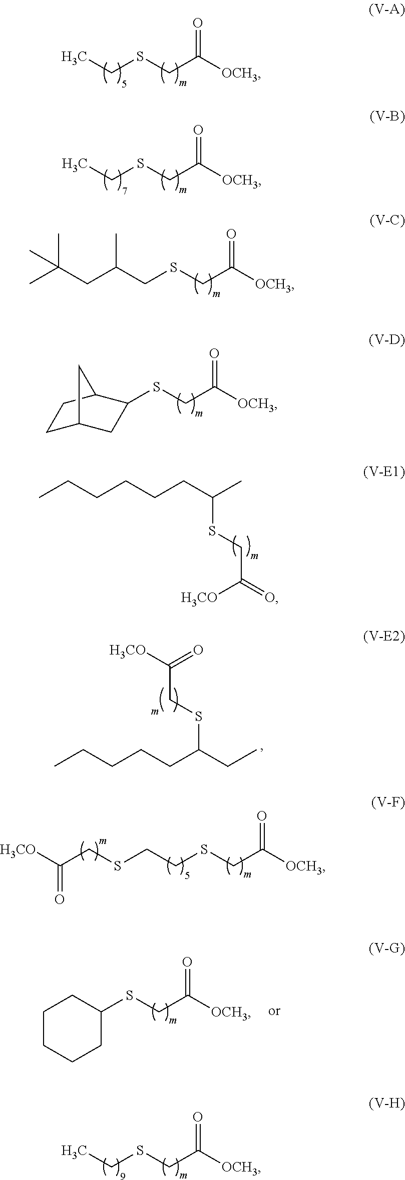

wherein each m of formulas (V-A)-(V-H) is, independently, 1 or 2.

Aspect 36. The process according to Aspect 30, wherein the thioether ester is represented by formula (V-I), formula (V-J), formula (V-K), formula (V-L), formula (V-M), formula (V-N), formula (V-O), formula (V-P), or combinations thereof:

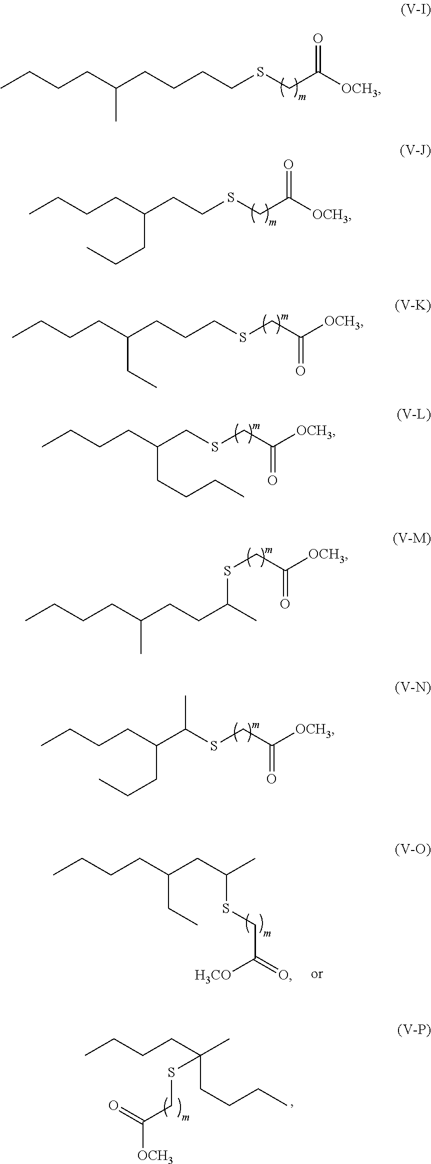

wherein each m of formulas (V-I)-(V-P) is, independently, 1 or 2.

Aspect 37. A solvent-free process for forming a functionalized organic sulfide, the solvent-free process comprising:
heating or maintaining, in a reactor, an olefin at a temperature in a range from about 20° C. to about 180° C., the olefin represented by formula (I):

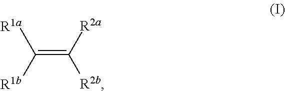

wherein each of R^{1a}, R^{1b}, R^{2a}, and R^{2b} of formula (I) is, independently, hydrogen, or a C1-C20 unsubstituted hydrocarbyl, or two or more of R^{1a}, R^{1b}, R^{2a}, or R^{2b} join together to form a C4-C30 monocyclic or polycyclic ring structure;
introducing a mercaptan to the olefin represented by formula (I) to form a mixture, the mercaptan represented by formula (II):

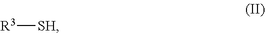

wherein R³ of formula (II) is a C1-C24 substituted hydrocarbyl; and wherein a molar ratio of the olefin represented by formula (I) to the mercaptan represented by formula (II) in the mixture is within a range from about 1:1 to about 10:1;
reacting the mixture, under conversion conditions, to form a conversion product effluent comprising a functionalized organic sulfide and unreacted olefin represented by formula (I), the conversion conditions optionally comprising:
   exposing the mixture to a first gas comprising air, O₂, or a combination thereof; and
   exposing the mixture to a second gas, the second gas being different from the first gas (for example, a non-reactive gas such as N₂);
separating the unreacted olefin from the conversion product effluent; and
combining the unreacted olefin with the mercaptan represented by formula (II) in the reactor.

Aspect 38. The process according to Aspect 37, wherein the process further comprises one or more implementations of the process according to any one of Aspects 1-36.

Aspect 39. A mining chemical collector comprising a functionalized organic sulfide described herein (for example, a functionalized organic sulfide formed by the process according to any one of Aspects 1-38).

Aspect 40. The mining chemical collector according to Aspect 39, wherein the functionalized organic sulfide comprises a thioether carboxylic acid described herein (for example, the thioether carboxylic acid according to any one of Aspects 31-32).

Aspect 41. A system for forming a functionalized organic sulfide, the system comprising:
a reactor configured to receive a first feed comprising an olefin (for example, olefin represented by formula (I)), to heat the first feed, to receive a second feed comprising a mercaptan (for example, mercaptan represented by formula (II)), to react a mixture comprising the first feed and the second feed under conversion conditions, and to discharge a conversion product effluent comprising a functionalized organic sulfide and unreacted olefin (for example, olefin represented by formula (I)); and
a separation unit configured to receive the conversion product effluent and to separate the conversion product effluent into a plurality of output streams, a first output stream of the plurality of output streams comprising the unreacted olefin.

Aspect 42. The system according to Aspect 41, wherein the system further comprises a line configured to transfer the first output stream from the separation unit to the reactor.

Aspect 43. The system according to any one of Aspects 41-42, wherein the reactor comprises a mixer, such as an impeller.

Aspect 44. The system according to any one of Aspects 41-43, wherein the mixer is configured to mix the first feed and the second feed.

Aspect 45. The system according to Aspect 44, wherein the mixer is further configured to mix the second feed with the first output stream comprising the unreacted olefin.

Aspect 46. The system according to any one of Aspects 41-45, wherein the reactor is further configured to expose the mixture comprising the first feed and the second feed to a first gas (for example, air, O₂, or a combination thereof), a second gas (for example, a non-reactive gas such as N₂), or both the first gas and the second gas.

Aspect 47. The system according to any one of Aspects 41-46, wherein the reactor is further configured to: expose the mixture comprising the first feed and the second feed to a first gas (for example, air, O₂, or a combination thereof); and expose the mixture comprising the first feed and the second feed to a second gas (for example, a non-reactive gas such as N₂).

Aspect 48. The system according to any one of Aspects 41-47, wherein the reactor comprises: a first inlet configured to receive the first feed; a second inlet configured to receive the second feed; a first outlet configured to discharge the conversion product effluent; or combinations thereof.

Aspect 49. The system according to Aspect 48, wherein the reactor further comprises: a third inlet configured to receive the unreacted olefin from the separation unit.

Aspect 50. The system according to any one of Aspects 41-49, wherein the separation unit comprises: an inlet configured to receive the conversion product effluent from the reactor; a first outlet configured to discharge the first output stream comprising the unreacted olefin; a second outlet configured to discharge a second output stream comprising the functionalized organic sulfide; or combinations thereof.

Aspect 51. The system according to any one of Aspects 41-50, wherein the reactor is further configured to:
expose the mixture comprising the first feed and the second feed to a partial pressure of a first gas comprising air, O₂, or a combination thereof, and
expose the mixture comprising the first feed and the second feed to a partial pressure of a second gas that is different from the first gas, the second gas comprising a non-reactive gas.

Aspect 52. A system for forming a functionalized organic sulfide, the system comprising:
a reactor;
a separation unit; and
a controller, the reactor and the separation unit coupled to the controller, the controller configured:
to cause the reactor to receive a first feed comprising an olefin, to heat the first feed, to receive a second feed comprising a mercaptan, to react a mixture comprising the first feed and the second feed under conversion conditions, and to discharge a conversion product effluent comprising a functionalized organic sulfide and unreacted olefin; and/or
to cause the separation unit to receive the conversion product effluent and to separate the conversion product effluent into a plurality of output streams, a first output stream of the plurality of output streams comprising the unreacted olefin.

Aspect 53. The system according to Aspect 52, wherein the system further comprises a line coupling the reactor to the separation unit, the line configured to transfer or recycle the first output stream from the separation unit to the reactor.

Aspect 54. The system according to Aspect 53, wherein the controller is further coupled to the line, the controller configured to open valves to transfer or recycle the first output stream from the separation unit to the reactor.

Aspect 55. The system according to any one of Aspects 52-54, wherein the controller is further configured to cause the reactor:
to expose the mixture comprising the first feed and the second feed to a first gas comprising air, O₂, or a combination thereof; and/or
to expose the mixture comprising the first feed and the second feed to a second gas that is different from the first gas, the second gas comprising a non-reactive gas.

Aspect 56. The system according to any one of Aspects 41-55, wherein the functionalized organic sulfide comprises a functionalized organic sulfide described herein (for example, the functionalized organic sulfide formed by the process according to any one of Aspects 1-38).

Aspect 57. A system for forming a functionalized organic sulfide, wherein the system is configured to perform any suitable process described herein (for example, the process according to any one of Aspects 1-38).

In the foregoing, reference is made to aspects of the disclosure. However, it should be understood that the disclosure is not limited to specific described aspects. Instead, any combination of the following features and elements, whether related to different aspects or not, is contemplated to implement and practice the disclosure. Furthermore, although aspects of the disclosure can achieve advantages over other possible solutions and/or over the prior art, whether or not a particular advantage is achieved by a given aspect is not limiting of the disclosure. Thus, the foregoing aspects, features, embodiments, implementations, and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a formulation, a composition, an element or a group of elements is preceded with the transitional phrase “comprising,” it is understood that we also contemplate the same formulation, composition or group of elements with transitional phrases “consisting essentially of,” “consisting of,” “selected from the group consisting of,” or “is” preceding the recitation of the formulation, composition, element, or elements and vice versa, for example, the terms “comprising,” “consisting essentially of,” “consisting of” also include the product of the combinations of elements listed after the term.

References cited herein are incorporated by reference herein in their entirety to indicate the state of the art as of their publication or filing date and it is intended that this information can be employed herein, if desired, to exclude specific aspects that are in the prior art.

For purposes of this present disclosure, and unless otherwise specified, all numerical values within the detailed description and the claims herein are modified by “about” or “approximately” the indicated value, and consider experimental error and variations that would be expected by a person having ordinary skill in the art. It will be further understood that there are a number of values disclosed therein, and that each value is also herein disclosed as “about” that particular value in addition to the value itself. In aspects, use of the term “about” can refer to ±15% of the stated value, ±10% of the stated value, +5% of the stated value, ±3% of the stated value, +2% of the stated value, or ±1% of the stated value.

For the sake of brevity, only certain ranges are explicitly disclosed herein. However, ranges from any lower limit can be combined with any upper limit to recite a range not explicitly recited, as well as, ranges from any lower limit can be combined with any other lower limit to recite a range not explicitly recited, in the same way, ranges from any upper limit can be combined with any other upper limit to recite a range not explicitly recited. Additionally, within a range includes every point or individual value between its end points even though not explicitly recited. Thus, every point or individual value can serve as its own lower or upper limit combined with any other point or individual value or any other lower or upper limit, to recite a range not explicitly recited. For example, by disclosing a temperature of from 70° C. to 80° C., an intent is to recite individually 70° C., 71° C., 72° C., 73° C., 74° C., 75° C., 76° C., 77° C., 78° C., 79° C., and 80° C., including any sub-ranges and combinations of sub-ranges encompassed therein such that any of the foregoing numbers can be used singly to describe an open-ended range or in combination to describe a close-ended range. Moreover, all numerical end points of ranges disclosed herein are approximate, unless excluded by proviso. As a representative example, if one or more operations in the processes described herein can be conducted at a temperature in a range from 10° C. to 75° C., this range should be interpreted as encompassing temperatures in a range from “about” 10° C. to “about” 75° C.

As used herein, the indefinite article “a” or “an” shall mean “at least one” unless specified to the contrary or the context clearly indicates otherwise. For example, aspects comprising “a mercaptan” include aspects comprising one, two, or more mercaptans, unless specified to the contrary or the context clearly indicates only one mercaptan is included.

When a compound is described herein such that a particular isomer, enantiomer, or diastereomer of the compound is not specified, for example, in a formula or in a chemical name, that description is intended to include each isomer and enantiomer of the compound described individual or in any combination. For example, any general structure, formula, or name presented is also intended to encompass all structural isomers, conformational isomers, regioisomers, stereoisomers (such as enantiomers, diastereomers, and other optical isomers whether in enantiomeric or racemic forms, as well as mixtures of stereoisomers, as the context permits or requires) that can arise from a particular set of substituents, unless indicated otherwise. Thus, a general reference to a compound includes all structural isomers unless specified to the contrary or the context clearly indicates otherwise. For example, reference to a hydrocarbon without specifying a particular isomer (such as butyl) expressly discloses all isomers thereof (such as n-butyl, iso-butyl, sec-butyl, and tert-butyl).

While the foregoing is directed to aspects of the present disclosure, other and further aspects of the disclosure can be devised without departing from the basic scope thereof, and the scope thereof is determined by the claims that follow.

What is claimed is:

1. A process for forming a functionalized organic sulfide, the process comprising:
introducing, to a reactor, an olefin represented by formula (I): 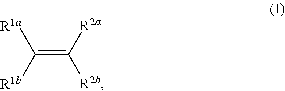
wherein each of R^{1a}, R^{1b}, R^{2a}, and R^{2b} of formula (I) is, independently, hydrogen, a C1-C20 unsubstituted hydrocarbyl, a C1-C20 substituted hydrocarbyl, or two or more of R^{1a}, R^{1b}, R^{2a}, and R^{2b} join to form a ring;
introducing a mercaptan to the olefin represented by formula (I) to form a mixture, the mercaptan represented by formula (II): 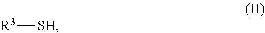
wherein R³ of formula (II) is a hydrogen, a C1-C24 substituted hydrocarbyl, or a C1-C24 unsubstituted hydrocarbyl;
reacting the mixture, under conversion conditions, to form a conversion product effluent comprising a functionalized organic sulfide and unreacted olefin represented by formula (I); and
separating the unreacted olefin from the conversion product effluent.

2. The process according to claim 1, wherein the process is solvent-free.

3. The process according to claim 1, wherein, after separating the unreacted olefin from the conversion product effluent, the process further comprises:
introducing the unreacted olefin to the reactor to react with the mercaptan represented by formula (II).

4. The process according to claim 1, wherein the conversion conditions comprise:
exposing the mixture to a first gas comprising air, O₂, or a combination thereof, and
exposing the mixture to a second gas that is different from the first gas, the second gas comprising a non-reactive gas.

5. The process according to claim 4, wherein:
a partial pressure of the first gas within the reactor is in a range from greater than 0 kPa to about 1,034 kPa; and
a total pressure within the reactor is in a range from greater than 0 kPa to about 2,068 kPa.

6. The process according to claim 4, wherein:
a partial pressure of the second gas within the reactor is in a range from about 1,034 kPa to less than 2,068 kPa; and
a total pressure within the reactor is in a range from greater than 0 kPa to about 2,068 kPa.

7. The process according to claim 4, wherein:
a partial pressure of the first gas is in a range from about 6.89 kPa to about 345 kPa; and
a partial pressure of the second gas is in a range from about 1,724 kPa to about 2,062 kPa.

8. The process according to claim 1, wherein the conversion conditions comprise a conversion temperature, the conversion temperature in a range from about 20° C. to about 165° C.

9. The process according to claim 8, wherein the conversion temperature is in a range from about 20° C. to about 110° C.

10. The process according to claim 1, wherein:
prior to introducing the mercaptan represented by formula (II) to the olefin represented by formula (I) to form the mixture, the process further comprises heating, or maintaining, the olefin represented by formula (I) at a temperature that is within 20° C. of a conversion temperature of the conversion conditions;
a molar ratio of the olefin represented by formula (I) in the mixture to the mercaptan represented by formula (II) in the mixture is in a range from about 1:1 to about 10:1 (olefin:mercaptan); or
a combination thereof.

11. The process according to claim 1, wherein, after separating the unreacted olefin from the conversion product effluent, an amount of unreacted mercaptan in the conversion product effluent is about 1 wt % or less based on a total wt % of the conversion product effluent after separating the unreacted olefin, the total wt % of the conversion product effluent after separating the unreacted olefin is 100 wt %.

12. The process according to claim 1, wherein:
at least one of R^{1a}, R^{1b}, R^{2a}, or R^{2b} of formula (I) is a C4-C16 unsubstituted hydrocarbyl;
R^{1a} and R^{2a} of formula (I) join to form a C5-C14 monocyclic or polycyclic ring structure;
at least one of R^{1a}, R^{1b}, R^{2a}, or R^{2b} of formula (I) contains at least one unsaturated carbon-carbon bond; or
combinations thereof.

13. The process according to claim 1, wherein the olefin represented by formula (I) comprises 1-butene, 1-pentene, 1-hexene, 1-heptene, 1-octene, 1-nonene, 1-decene, 1-undecene, 1-dodecene, cyclohexene, diisobutylene, trans-2-octene, norbornene, 1,5-hexadiene, 4-methyl-1-pentene, 3-methyl-1-pentene, 5-methyl-1-nonene, 3,5,5-trimethyl-1-hexene, 1,5-cyclooctadiene, 2-ethyl-1-hexene, 2-butyl-1-octene, 3,7-dimethyl-1-octene, a propylene oligomer, an isobutylene oligomer, a branched C10 monoolefin, or combinations thereof.

14. The process according to claim 1, wherein:
R³ of formula (II) comprises a C2-C14 substituted hydrocarbyl;
R³ of formula (II) comprises a hydroxyl group, a carboxylic acid group, an ester group, an amine group, an amide group, a nitrile group, a cyano group, or a silicon-containing group; or
a combination thereof.

15. The process according to claim 1, wherein the mercaptan represented by formula (II) comprises beta-mercaptoethanol, 3-mercaptopropanol, 1-mercapto-2-propanol, 4-sulfanylbutan-1-ol, thioglycolic acid, 3-mercaptopropanoic acid, methylthioglycolate, methyl-3-mercaptopropionate, or combinations thereof.

16. A solvent-free process for forming a functionalized organic sulfide, the solvent-free process comprising:
heating or maintaining, in a reactor, an olefin at a temperature in a range from about 20° C. to about 180° C., the olefin represented by formula (I): 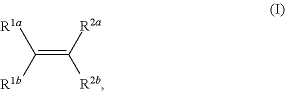
wherein each of R^{1a}, R^{1b}, R^{2a}, and R^{2b} of formula (I) is, independently, hydrogen, or a C1-C20 unsubstituted hydrocarbyl, or two or more of R^{1a}, R^{1b}, R^{2a}, and R^{2b} join to form a ring;
introducing a mercaptan to the olefin represented by formula (I) to form a mixture, the mercaptan represented by formula (II): 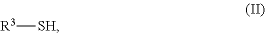
wherein R³ of formula (II) is a C1-C24 substituted hydrocarbyl; and
wherein a molar ratio of the olefin represented by formula (I) to the mercaptan represented by formula (II) in the mixture is within a range from about 1:1 to about 10:1;
reacting the mixture, under conversion conditions, to form a conversion product effluent comprising a functionalized organic sulfide and unreacted olefin represented by formula (I), the conversion conditions comprising:
exposing the mixture to a first gas comprising air, O₂, or a combination thereof; and
exposing the mixture to a second gas, the second gas being different from the first gas, the second gas comprising a non-reactive gas;
separating the unreacted olefin from the conversion product effluent; and
combining the unreacted olefin with the mercaptan represented by formula (II) in the reactor.

17. The solvent-free process according to claim 16, wherein the functionalized organic sulfide comprises a thioether carboxylic acid, a thioether alcohol, a thioether ester, or combinations thereof.

18. A system for forming a functionalized organic sulfide, the system comprising:
a reactor;
a separation unit; and
a controller, the reactor and the separation unit coupled to the controller, the controller configured:
to cause the reactor to receive a first feed comprising an olefin, to heat the first feed, to receive a second feed comprising a mercaptan, to react a mixture comprising the first feed and the second feed under conversion conditions, and to discharge a conversion product effluent comprising a functionalized organic sulfide and unreacted olefin; and
to cause the separation unit to receive the conversion product effluent and to separate the conversion product effluent into a plurality of output streams, a first output stream of the plurality of output streams comprising the unreacted olefin.

19. The system according to claim 18, wherein the system further comprises a line coupling the separation unit to the reactor, the line configured to transfer the first output stream from the separation unit to the reactor.

20. The system according to claim 18, wherein the controller is further configured to cause the reactor:
to expose the mixture comprising the first feed and the second feed to a first gas comprising air, O₂, or a combination thereof; and
to expose the mixture comprising the first feed and the second feed to a second gas that is different from the first gas, the second gas comprising a non-reactive gas.
